(19) United States
(12) Patent Application Publication
(10) Pub. No.: US 2021/0142879 A1
(43) Pub. Date: May 13, 2021

(54) SYSTEMS AND METHODS FOR COMMUNICATING A DOSE

(71) Applicant: Novo Nordisk A/S, Bagsvaerd (DK)

(72) Inventors: Pete Brockmeier, Copenhagen V (DK); Michael Friis, Copenhagen N (DK); Alan John Michelich, Seattle, WA (US); Tinna Bjoerk Aradottir, Copenhagen (DK); Henrik Bengtsson, Taastrup (DK)

(21) Appl. No.: 16/488,281

(22) PCT Filed: Feb. 6, 2018
(86) PCT No.: PCT/EP2018/052862

§ 371 Date: Aug. 23, 2019

(30) Foreign Application Priority Data
Feb. 23, 2017 (EP) 17157529.3

(51) Int. Cl. G16H 20/30; G16H 10/60; G16H 40/67

(52) U.S. Cl. CPC G16H 20/30; G16H 40/67; G16H 10/60

(57) ABSTRACT

Systems and methods for communicating a dose history configured for representing a central tendency and a variability of a distribution of injections with a blood glucose regulating medicament. The device (250) is adapted for performing the method of, obtaining one or more qualified groups of injection events within a distribution of injection events, wherein each qualified group of injection events comprises a group-time indicator (243); for each respective qualified group of injection events (241) within the set of qualified groups of injection events (240): (i) determining, on a temporal basis, a subset of grouped medicament records (242) corresponding to the respective qualified group of injection events (241), using the group-time indicator (243), (ii) processing the subset of grouped medicament records (242) of the respective qualified group of injection events to obtain display data (249) configured to represent a measure of central tendency (244, 246) and a measure of variability (245, 247) related to the relative time (237); and communicating the display data (249).

### TECHNICAL FIELD

The present disclosure relates generally to systems and methods for communicating a dose history configured for representing a central tendency and a variability of a distribution of injections with a blood glucose regulating medicament applied by a subject with a treatment regimen.

### BACKGROUND

Type 2 diabetes mellitus is characterized by progressive disruption of normal physiologic insulin secretion. In healthy individuals, basal insulin secretion by pancreatic β cells occurs continuously to maintain steady glucose levels for extended periods between meals. Also in healthy individuals, there is prandial secretion in which insulin is rapidly released in an initial first-phase spike in response to a meal, followed by prolonged insulin secretion that returns to basal levels after 2-3 hours.

Insulin is a hormone that binds to insulin receptors to lower blood glucose by facilitating cellular uptake of glucose, amino acids, and fatty acids into skeletal muscle and fat and by inhibiting the output of glucose from the liver. In normal healthy individuals, physiologic basal and prandial insulin secretions maintain euglycemia, which affects fasting plasma glucose and postprandial plasma glucose concentrations. Basal and prandial insulin secretion is impaired in Type 2 diabetes and early post-meal response is absent. To address these adverse events, subjects with Type 2 diabetes are provided with insulin medicament treatment regimens. Subjects with Type 1 diabetes are also provided with insulin medicament treatment regimens. The goal of these insulin medicament treatment regimens is to maintain a desired fasting blood glucose target level that will minimize estimated risk of hypo- and hyper-glycaemia. In recent years subjects with Type 2 diabetes have also been treated with liraglutide, long-acting glucagon-like peptide-1 receptor agonist, as an injectable prescription medicine that may regulate and improve blood sugar, and it should be used along with diet and exercise.

Traditional insulin medicament delivery systems have included the use of pump systems that provide a frequent recurrent dosage of insulin medicament. Additional types of delivery systems have been developed, such as insulin pens, which can be used to self-administer insulin medicament treatment regimens in the form of less frequent insulin medicament injections or injections with other types of blood glucose regulating medicaments. A common approach to Type 1 and Type 2 diabetes using such delivery systems is to inject a single short acting insulin medicament (bolus) dosage in a prescribed insulin regimen for the subject in response to or in anticipation of a meal event. In such approaches, the subject injects the short acting insulin medicament dosage shortly before or after one or more meals each day to lower glucose levels resulting from such meals.

A recent development for injection devices, is the development of injector systems which are capable of storing dose history (dose size and time), and subsequently sending historical dose data to a mobile phone or computer system. There is a need to effectively visualize this data. The data can be visualized in combination with historical glucose data, in order to draw conclusions about the appropriateness of the dose regimen for desired glucose control.

A common method of representation of glucose data for viewing by a health care provider or patient is the Ambulatory Glucose Profile (AGP), which was developed by clinicians to demonstrate the median level of glucose control as well as an index of variability in control at each hour of a “standard day.” The ability to show both an average glucose value, as well as variability, is an important element of AGP. If the average glucose is higher than the target range, but the variability is also very high, it may be dangerous to address this by simply increasing insulin dose size, as hypoglycaemia could result. Furthermore, it is known that the existence of high variability in blood glucose can be detrimental, even with an average within range. US 2014/0206970 discloses a method of generating an ambulatory glucose profile window including a graphical display of the glucose data across a modal day.

The visual display presents a modal day (also called standard day, average day) in which all collected data over multiple days are collapsed and plotted according to time (without regard to date) as if they occurred over 24 h, starting and ending at midnight. Smoothed curves representing the median (50th), 25th, and 75th (IQR) and 10th and 90th frequency percentiles define the 24 h AGP, as further described in Journal Diabetes Science Technology, March 2013, Volume 7, Issue 2: pages 562-578.

While clinicians can use this type of visualized data to make some conclusions about the suitability of the current insulin regime being used by the patient, the glucose curve is the output of a number of inputs. An important input, for a diabetic patient, is injections with blood glucose regulating medicaments.

Doug Kanter represented in a final project for the Data Representation class at ITP, an insulin on board profile showing the accumulated insulin on board delivered by an insulin pump and the corresponding glucose data. The project was published on: https://dougkanter.wordpress.com/2012/05/14/insulin-on-board-data-rep-final-project/. The link was retrieved on 14 Feb. 2017.

Medtronic represented in a Report Reference Guide for CareLink pro, which is a therapy management software for diabetes, that the basal and the bolus infusion rate can be shown along with glucose data. The software is developed for handling insulin data from a pump.

The guide was published on: https://www.medtronicdiabetes.com/sites/default/files/library/download-library/user-guides/carelink-v3_0/en_carelink_pro_report_ref_guide.pdf. The link was retrieved on 14 Feb. 2017.

WO 2015/047870 discloses a system for delivering and recording a dose of a medicament to a patient, WO 2016/007935 discloses methods, systems and devices for administering a medicament to a patient. The system includes an injection pen device in wireless communication with a mobile communication device. The device comprises an electronics unit in communication with a sensor unit to process a detected dispensed dose and time data associated with a dispensing event, and to wirelessly transmit the dose data to a user's device. The mobile communication device provides a software application to provide the user with health information using the processed data.

EP 2774641 B1 discloses an arrangement for administering a selected dosage of insulin. The arrangement comprises a sensor for contactless sensing of an adjusted dose. US 2013/0079727 discloses an application assembly comprising means for determining and registering the time and/or date and means for determining the selected and administered dosage. The date and/or the time may be transmitted to a receiver by means of a transmitter together with the signal of the applied amount of the medicament. The transmission can e.g. be via Bluetooth to a cell phone. The assembly may be provided with a display for showing warnings, transmission data, status information and the like. This facilitates the handling.

US 2006/0272652 identifies a need to provide both diabetes patients and medical professionals with an interactive visual teaching tool that illustrates the effects of certain intakes and events on blood glucose levels and present this information in an easy-to-read and understandable user format. The document discloses a screen where a doctor manipulate and view screen has been displayed. The doctor manipulate and view screen includes an insulin delivery graph, a carbohydrate ingested graph, and a blood glucose level graph. The timeframe illustrated in the doctors manipulate and view screen is in a modal mode of one day. Each of the days having readings displayed in the doctor manipulate and view screen are displayed in a different color or with a different width/typeface. Illustratively, one line represents Monday, a second line represents Tuesday, and a third line represents Wednesday. This view allows a doctor utilizing the virtual patient software to see multiple days of readings for a specific patient and to determine if a time frame specific problem is occurring. The insulin delivery graph is illustrated by rectangles indicating time of injection and magnitude of injected medicament.

US 2016/0098848 discloses a presentation template including data visualization corresponding to at least one signal trace of measured glucose values with respect to time, and may further include ingestion, and an indicator of insulin intake may be displayed above the signal trace, whereby the indicator of insulin intake may be read as pushing down on the signal trace. In both CGM data (data relating to continuous glucose monitoring) and SMBG data (data relating to self-monitored blood glucose), stacking icons corresponding to boluses and meal intake can help the user visualize the amount of each administered. In multi-day views of the same, stacking icons corresponding to boluses and meal intake (or providing histogram views of the same) may be accomplished by averaging the boluses and meal intake over the course of the multi-day time period.

Eventhough prior art allegedly discloses the possibility of stacking icons or producing histograms corresponding to boluses in a multiday view, by averaging the boluses a user will not be presented with information indicating the rhythm in which the injection events occur, and how much this rhythm may vary. A treatment regimen specifies instructions of how and when to apply the injections with the blood regulating medicament. When the regimen is applied, different patterns will emerge in the distribution of injection events, and the patterns will depend on the subjects rhythms in activity and the prescribed treatment regimen. In order to understand retrospectively how the treatment regimen is applied it is necessary to obtain and communicate these patterns in a way which indicates the rhythm of how the injections are applied. As appears this issue has not been addressed by the prior art.

Having regard to the above, it is an object of the present invention to provide a device or a system and a method for communicating a dose history configured for representing a central tendency and a variability of a distribution of injections with a blood glucose regulating medicament applied by a subject with a treatment regimen, obtaining one or more qualified groups of injection events within the distribution of injection events, and thereby enabling a communication of information relating to the time related properties of the distribution.

It is a further object of the invention to provide a device or a system and a method for automatically identifying a group of injection events as a pattern within the distribution of injection events and thereby enabling the communication of information relating to a rhythm of how the treatment regimen is applied, and communication of information relating to the time related properties of the identified groups.

It is a further object of the invention to provide a device or a system and a method for identifying a group of injection events as a pattern within the distribution of injection events, wherein the identification is based on pre-defined instructions on which time intervals are of interest and thereby enabling communication of information relating to the time related properties of the identified group.

It is a further object of the invention to provide a device or a system and a method for, in combination with a dose history, further communicating glucose measurements of the subject, and thereby enabling a communication of information of relation between glucose data and the distribution of injection event within a time period.

It is a further object of the invention to provide a device or a system and a method for, in combination with a dose history, further communicating a life-style event history representing an average and a variability of a distribution of life-style related events within the time course, which the subject has engaged in, and thereby improving the possibilities of communicating the relation between the events the subject engages in.

### SUMMARY

In the disclosure of the present invention, embodiments and aspects will be described, which will address one or more of the above objects or which will address objects apparent from the below disclosure as well as from the description of exemplary embodiments.

In a first aspect is provided, a device for communicating a dose history configured for representing a central tendency and a variability of a distribution of injections with a blood glucose regulating medicament applied by a subject with a treatment regimen;
the device comprises one or more processors and a memory, the memory storing instructions that, when executed by the one or more processors, perform a method of:
obtaining a first data set from one or more injection devices used by the subject to apply the treatment regimen, the first data set comprising a plurality of medicament records taken over a time course, each respective medicament record in the plurality of medicament records comprising:
   (i) a respective medicament injection event including an amount of medicament injected into the subject using a respective injection device in the one or more injection devices,
   (ii) a corresponding electronic injection event timestamp within the time course that is automatically generated by the respective injection device upon occurrence of the respective medicament injection event;
creating a plurality of consecutive time windows within the time course, wherein each time window is of the same fixed duration,
   for each respective time window, obtaining a subset of medicament records, and thereby obtaining a plurality of subsets of medicament records, wherein each respective subset of medicament records comprises a number of medicament records from the first data set, and wherein each respective medicament record within the respective subset of medicament records have a timestamp in the respective time window;
for each respective medicament record, within each subset of medicament records of the plurality of subsets of medicament records, assigning a corresponding relative time being the relative time within the time window;
selecting a set of subsets of medicament records from the plurality of subsets of medicament records and thereby obtaining a set of selected subsets of medicament records comprising a number of selected subsets of medicament records representing a distribution of injection events within an interval corresponding to the fixed duration of the time windows;
obtaining one or more qualified groups of injection events within the distribution of injection events, and thereby obtaining a set of qualified groups of injection events, wherein each qualified group of injection events comprises a group-time indicator;
for each respective qualified group of injection events within the set of qualified groups of injection events:
   (i) determining, on a temporal basis, a subset of grouped medicament records corresponding to the respective qualified group of injection events, using the group-time indicator and the relative time of each of the medicament records in each selected subset of medicament records of the set of selected subsets of medicament records, and thereby obtaining a subset of grouped medicament records,
   (ii) processing the subset of grouped medicament records of the respective qualified group of injection events and evaluating a measure of central tendency and a measure of variability of the subset of grouped medicament records to obtain display data configured to represent a measure of central tendency and a measure of variability of injection events within the respective qualified group of injection events, wherein the measure of central tendency and the measure of variability is related to the relative time; and
communicating the display data comprising the evaluated measure of central tendency and the evaluated measure of variability of the subset of grouped medicament records to (i) the subject, (ii) to a health care provider, or (iii) to the user of the device, and thereby communicating the central tendency and the variability of the distribution of injection events representing the set of selected subsets of medicament records.

Hereby is provided a device adapted for performing a method for communicating a dose history configured for representing a central tendency and a variability of a distribution of injections with a blood glucose regulating medicament applied by a subject with a treatment regimen, obtaining one or more qualified groups of injection events within the distribution of injection events, and thereby improving the possibility of communicating the rhythm of how the treatment regimen is applied, as the method provides grouped injection data and a central tendency and variability, on a temporal basis, relating to that group of injections. As appears the distribution of injection events representing the set of selected subsets of medicament records, comprises one or more qualified groups of injection events, and may therefore be a multimodal distribution. As appears, a major advantage of the device is that it provides the ability to obtain and communicate, on a group-basis in an average day, modal day or standard day, data configured for illustrating how a patient injects insulin over a period of time, as well as the variability of time with which a patient injects insulin within these groups of injections, i.e., in which rhythm the injections are applied. The grouping of injection events and visualisation of injection doses gives an HCP a quick summary of how the patient has, in a standard day, applied insulin injections in a way that visually compliments the way that AGP gives a picture of the standard day of blood glucose. In effect, the device is adapted for performing a method of obtaining injection event data, analysing and qualifying groups of injection events on a temporal basis, and thereby enabling the creation of data structures comprising relevant parameters and data for the qualified groups, and wherein the data structures are adapted for communicating at list information relating to the temporal appearance, i.e., the time related appearance of qualified groups within the distribution. The memory comprises an enriched display data structure which easily can be communicated to provide the above mentioned advantages. The central tendency can be defined as the tendency of quantitative data to cluster around some central value. The closeness with which the values surround the central value is commonly quantified using the standard deviation. The variability, is in contrast to the central tendency the variability or spread in a variable or a probability distribution, and indicates how much observations in a data set vary. The evaluating a measure of central tendency and the evaluating a measure of variability of the subset of grouped medicament records, can be on a temporal basis, in addition it can also be on an amount of injected insulin basis.

In a further aspect of the device, the step (ii) of processing the subset of grouped medicament records of the respective qualified group of injection events to obtain display data further comprises: processing the subset of grouped medicament records of the respective qualified group of injection events to obtain display data configured to represent a measure of central tendency and a measure of variability of injection events within the respective qualified group of injection events, wherein the measure of central tendency and the measure of variability is related to the amount of medicament injected into the body.

Hereby the device is adapted to provide data structures further adapted for communicating information of a property relating to the dose size and the variation of the dose within the qualified group.

In a further aspect of the device, the step of obtaining one or more qualified groups of injection events within the distribution of injection events, and thereby obtaining a set of qualified groups of injection events further comprises:
estimating a probability density function of the distribution of injections events, by using the set of selected subsets of medicament records;
identifying one or more groups of injection events within the distribution of injection events, by using the probability density function, wherein each of the identified groups of injection events are identified by a peak indicating a local maximum of the probability density function, wherein the identified peak comprises a peak value and a corresponding peak time, and thereby obtaining a set of identified groups of injection events;
responsive to identifying one or more groups of injection events, for each respective identified group of injection events within the set of identified groups of injection events, evaluating whether the respective identified group of injection events is qualified to be communicated, and thereby obtaining a set of qualified groups of injection events; and
wherein the step (i) of determining the subset of grouped medicament records, for each respective qualified group of injection events further comprises using the peak-time as the group-time indicator.

Hereby is provided a device further adapted for automatically identifying a group of injection events as a pattern within the distribution of injection events and thereby enabling the communication of information relating to a rhythm of how the treatment regimen is applied, and communication of information relating to the time related properties of the identified groups, i.e., how often are injections applied and what is the magnitude of variation of the applied injection.

In a further aspect of the device, the step of evaluating whether the respective identified group of injection events is qualified to be communicated comprises evaluating whether a function of the peak value is below a pre-defined threshold for qualification. The function of the peak value may be a linear function defined by a constant of proportionality and an off-set, and in a simle case the constant of proportionality is 1 and the off-set is zero. In another example the function may be a polynomial of a higher degree, e.g., a function of second degree.

Hereby the identified group can be evaluated to be a qualified group if the peak value is above the pre-defined threshold, and not a qualified group if the peak value is below the pre-defined threshold.

In a further aspect the device is adapted for obtaining a qualified group in an alternative way, wherein the step of obtaining one or more qualified groups of injection events within the distribution of injection events, and thereby obtaining a set of qualified groups of injection events, further comprises:
obtaining a set of pre-defined time ranges, wherein each of the pre-defined time ranges within the set of pre-defined time ranges are defined within the fixed duration of the time windows, and wherein none of the pre-defined time ranges are overlapping another pre-defined time range within the set of pre-defined time ranges;
for each pre-defined time range:
   obtaining an identified group of injection events and thereby obtaining a set of identified groups of injection events,
   evaluating whether the identified group of injection events is a qualified group of injection events, and thereby obtaining a set of qualified groups of injection events; and
wherein the step (i) of determining the subset of grouped medicament records, for each respective qualified group of injections events, further comprises using the pre-defined time range as the group-time indicator.

Hereby is provided a device further adapted for identifying a group of injection events as a pattern within the distribution of injection events, wherein the identification is based on pre-defined instructions on which time intervals are of interest and thereby enabling communication of information relating to the time related properties of the identified group.

In a further aspect of the device, for each pre-defined time range, the step of obtaining an identified group of injection events further comprises:
determining, on a temporal basis, a subset of identified medicament records of the medicament records within the set of selected subsets of medicament records, wherein the medicament records within the subset of identified medicament records have a relative time within the respective pre-defined time range; and
wherein the step of evaluating whether the identified group of injection events is a qualified group of injection events further comprises:
evaluating the number of medicament records within the subset of identified medicament records,
evaluating whether the number of medicament records is above a pre-defined threshold, wherein the identified group is qualified, or below a pre-defined threshold, wherein the identified group is disqualified;
responsive to the identified group is qualified characterizing the identified group of injection events as a qualified group of injection events.

Hereby an identified group can be qualified based on the number of medicament records within the group. If the number of medicament records is above the pre-defined threshold the identified group can be evaluated as qualified, and vice versa if the number is below.

In a further aspect of the device, for each pre-defined time range, the step of evaluating whether the identified group of injection events is a qualified group of injection events further comprises:
obtaining a user input indicating whether the pre-defined time range is qualified or disqualified; and
responsive to the obtained user input indicates that the pre-defined time range is qualified, characterizing the identified group of injection events as a qualified group of injection events.

Hereby an identified group can be characterized qualified based on a user input. If the user, e.g., has additional knowledge and therefore decides that he will be able to see the identified group in question in a displayed configuration.

In a further aspect of the device, for each pre-defined time range, the step of evaluating whether the identified group of injection events is a qualified group of injection events further comprises:
obtaining a pre-defined input indicating whether the pre-defined time range is qualified or disqualified; and
responsive to the obtained pre-defined input indicates that the pre-defined time range is qualified, characterizing the identified group of injection events as a qualified group of injection events.

Hereby an identified group can be characterized qualified based on a pre-defined input. If the user or a health care person (HCP), e.g., has additional knowledge to the injection data and he decides that it will be relevant always to see the identified groups in question in a displayed configuration.

In a further aspect of the device, the step (ii) of processing the subset of grouped medicament records, for each respective qualified group of injection events, to obtain display data configured to represent a central tendency and a variability of injection events comprises: evaluating a measure of central tendency for the relative time and the amount of medicament injected into the body, and evaluating a measure of variability for the relative time and the amount of medicament injected into the body.

In a further aspect of the device, the step of determining a measure of central tendency comprises evaluating a median, and determining a measure of variability comprises evaluating an upper and a lower percentile.

In a further aspect of the device, the step of determining a measure of central tendency comprises evaluating a mean, and determining a measure of variability comprises evaluating a standard deviation.

In a further aspect of the device, the display data comprises the data configured to represent an average and a variability of injection events within the respective qualified group of injection events.

In a further aspect of the device, the display data comprises the set of qualified groups of injection events.

In a further aspect of the device, the step (ii) of processing the subset of grouped medicament records, for each respective qualified group of injection events, to obtain display data, further comprises:
associating with each qualified group of injection events:
   a shape data structure, configured for representing the central tendency and the variability of the subset of grouped medicament records corresponding to the respective qualified group of injection events, wherein the shape data structure comprises:
      a central tendency data-structure comprising a central tendency polygon configured for visualizing a polygon with a two-dimensional shape indicating the measure of central tendency,
      a variability data-structure comprising a variability polygon configured for visualizing a polygon with a two-dimensional shape identifying the measure of variability.

Hereby is provided a device adapted for graphically communicating average and variability of each of the qualified groups, as a polygon with a two-dimensional.

In a further aspect of the device, the step (ii) of processing the subset of grouped medicament records, for each respective qualified group of injection events, to obtain display data, further comprises:
associating with each qualified group of injection events a table data structure comprising:
   a qualified group identification,
   a median time and a time variation based on the relative time, of the subset of grouped medicament records, and
   a median dose and a dose variation based on the amount of injected medicament (226) of the subset of grouped medicament records

Hereby is provided a device adapted for communicating average and variability of each of the qualified groups in a table.

In a further aspect of the device, the step (ii) of processing the subset of grouped medicament records, for each respective qualified group of injection events, to obtain display data, further comprises:
obtaining display data configured to represent a frequency indicator indicating the frequency of an injection event of the respective qualified group of injection events, wherein the frequency indicator is a function of:
   the number of medicament records in the subset of grouped medicament records over the total number of medicament records in all subsets of grouped medicament records in the set of qualified groups of injection events, or
   the number of medicament records in the subset of grouped medicament records over the total number of medicament records of all selected subsets medicament records in the set of selected subsets of medicament records;
thereby enabling an indication on the frequency of injections in the respective qualified group of injection events, and wherein the display data further comprises the frequency indicator.

Hereby the obtained display data enables an indication of the frequency of injections in the respective qualified group of injection events.

In a further aspect of the device, the step (ii) of processing the subset of grouped medicament records, for each respective qualified group of injection events, to obtain display data, further comprises:
obtaining display data configured to indicate a frequency of an injection event of the respective qualified group of injection events;
normalizing the probability density function and the peak value to obtain a normalized peak value, whereby the normalized peak value indicates the frequency of an injection event of the respective qualified group of injection events, and wherein the display data comprises the normalized peak value.

Hereby the obtained display data structure enables an indication on the frequency of injections in the respective qualified group of injection events by using the normalized probability distribution.

In a further aspect of the device, the treatment regimen comprises a GLP-1 receptor agonist dosage regimen, with a medicament comprising a GLP-1 receptor agonist.

In a further aspect of the device, the treatment regimen comprises a bolus insulin medicament dosage regimen with a short acting insulin medicament.

In a further aspect of the device, each respective medicament record in the plurality of medicament records further comprises:
(iii) a corresponding type of medicament injected into the subject; and
wherein the set of selected subsets of medicament records comprises the same type of medicament and thereby represents a distribution of injection events corresponding to the respective type of medicament; and
wherein the step (ii) of processing the subset of grouped medicament records, for each respective qualified group of injection events, to obtain display data, further comprises obtaining display data configured to represent the respective type of medicament. Hereby the medicament records enables that data relating to different types of medicament can be distinguished and processed separately.

In a further aspect of the device, the treatment regimen comprises a bolus insulin medicament dosage regimen with a short acting insulin medicament and a basal insulin medicament dosage regimen with a long acting insulin medicament.

In a further aspect, the device further comprises a display configured for representing a first coordinate system, and wherein the step of communicating display data further comprises:
displaying the obtained display data, configured to represent a central tendency and a variability of injection events within the respective qualified group of injection events, in the first coordinate system on the display, the first coordinate system comprises a first axis and a second axis:
wherein the first coordinate system is adapted to represent the central tendency and the variability of the injection events; and
wherein the first axis represents the relative time and are defined within the interval defined by the time window, and wherein the second axis represents the amount of injected medicament.

In a further aspect of the device, the method further comprises:
obtaining a second data set, wherein the second data set comprises a plurality of autonomous glucose measurements of the subject within the time course and, for each respective autonomous glucose measurement in the plurality of autonomous glucose measurements, a glucose measurement timestamp representing when the respective measurement was made; and
for each respective time window, creating a set of glucose measurements, and thereby creating a plurality of sets of glucose measurements, and wherein each glucose measurement within the respective set of glucose measurements have a timestamp in the respective time window;
for each respective glucose measurement, associating a corresponding relative time being the relative time within the time window, whereby the plurality of sets of glucose measurements are representing a distribution of glucose measurements within the time window;
calculating, for the plurality of sets of glucose measurements, the central tendency and the variability as a function of the relative time,
wherein the display data further comprises the plurality of sets of glucose measurements, the corresponding relative time, and the calculated central tendency and the variability as a function of the relative time.

Hereby is provided a device for, in combination with a dose history, further communicating glucose measurements of the subject, and thereby enabling a communication of information of relation between glucose data and the distribution of injection event within a time period.

In a further aspect of the device, the display is adapted to represent a first and a second coordinate system each comprising a first axis and a second axis, and
wherein the first coordinate system is adapted to represent the central tendency and the variability of the injection events; and
wherein the second coordinate system is adapted to represent the central tendency and the variability of the glucose data; and
wherein the step of communicating display data further comprises:
displaying the obtained display data, configured to represent an central tendency and a variability of injection events within a respective qualified group of injection events, in the first coordinate system on the display, and
displaying the obtained display data, comprising an central tendency and a variability of the plurality of sets of glucose measurements as a function of time, in the second coordinate system on the display, and
wherein, for the first coordinate system, the second axis represents the amount of injected medicament, and wherein, for the second coordinate system, the second axis represents a blood glucose concentration, and wherein the first axis of each coordinate systems represent the relative time and are defined within the interval defined by the time window.

Hereby injection data and blood glucose data can be presented in separate, but comparable coordinate systems.

In a further aspect, the device is further adapted for communicating a life-style event history representing an central tendency and a variability of a distribution of life-style related events within the time course, which the subject has engaged in, wherein the method further comprises:
obtaining a third data set from one or more wearable life-style measurement devices used by the subject to acquire life-style data, the third data set comprises a plurality of life-style data records over the time course, each respective life-style data record in the plurality of life-style data records comprises:
   (i) a respective life-style event including a measure of intensity indicating the effect on the subject using the respective measurement device,
   (ii) a corresponding electronic life-style event timestamp within the time course that is automatically generated by the respective life-style measurement device upon occurrence of the respective life-style related event, or by user actuation of the respective life-style measurement device, or a begin timestamp and an end timestamp indicating the beginning and the ending time of the life-style event engaged in by the subject;
   for each respective time window, obtaining a subset of life-style data records, and thereby obtaining a plurality of subsets of life-style data records, wherein each respective subset of life-style data records comprises a number of life-style data records from the third data set, and wherein each respective life-style data record within the respective subset of life-style data records have a timestamp in the respective time window;
for each respective life-style data record, within each subset of life-style data records of the plurality of subsets of life-style data records, assigning a corresponding relative time being the relative time within the time window;
selecting a set of subsets of life-style data records from the plurality of subsets of life-style data records and thereby obtaining a set of selected subsets of life-style data records comprising a number of selected subsets of life-style data records representing a distribution of life-style events within an interval corresponding to the fixed duration of the time windows;
obtaining one or more qualified groups of life-style events within the distribution of life-style events, and thereby obtaining a set of qualified groups of life-style events, wherein each qualified group of life-style events comprises a group-time indicator;
for each respective qualified group of life-style events within the set of qualified groups of life-style events:
   (i) determining, on a temporal basis, a subset of grouped life-style data records corresponding to the respective qualified group of life-style events, using the group-time indicator and the relative time of each of the life-style data records in each selected subset of life-style data records of the set of selected subsets of life-style data records, and thereby obtaining a subset of grouped life-style data records,
   (ii) processing the subset of grouped life-style data records of the respective qualified group of life-style events to obtain display data further configured to represent a measure of central tendency and a measure of variability of life-style events within the respective qualified group of life-style events, wherein the measure of central tendency and the measure of variability is related to the relative time and/or the measure of intensity; and
communicating the display data to (i) the subject, (ii) to a health care provider, or (iii) to the user of the device, and thereby communicating the central tendency and the variability of the injection events.

Hereby is provided a device or a system for, in combination with a dose history, further communicating a life-style event history representing an average and a variability of a distribution of life-style related events within the time course, which the subject has engaged in, and thereby improving the possibilities of communicating the relation between the events the subject engages in.

In a further aspect is provided a computer implemented method for communicating a dose event history representing a central tendency and a variability of a distribution of a distribution injections with a blood glucose regulating medicament applied by a subject with a treatment regimen;
using a device comprising one or more processors and a memory, the memory storing instructions that, when executed by the one or more processors, perform a method of:
obtaining a first data set from one or more injection devices used by the subject to apply the treatment regimen, the first data set comprising a plurality of medicament records taken over a time course, each respective medicament record in the plurality of medicament records comprising:
   (i) a respective medicament injection event including an amount of medicament injected into the subject using a respective injection device in the one or more injection devices,
   (ii) a corresponding electronic injection event timestamp within the time course that is automatically generated by the respective injection device upon occurrence of the respective medicament injection event;
creating a plurality of consecutive time windows within the time course, wherein each time window is of the same fixed duration,
   for each respective time window, obtaining a subset of medicament records, and thereby obtaining a plurality of subsets of medicament records, wherein each respective subset of medicament records comprises a number of medicament records from the first data set, and wherein each respective medicament record within the respective subset of medicament records have a timestamp in the respective time window;
for each respective medicament record, within each subset of medicament records of the plurality of subsets of medicament records, assigning a corresponding relative time being the relative time within the time window;
selecting a set of subsets of medicament records from the plurality of subsets of medicament records and thereby obtaining a set of selected subsets of medicament records comprising a number of selected subsets of medicament records representing a distribution of injection events within an interval corresponding to the fixed duration of the time windows;
obtaining one or more qualified groups of injection events within the distribution of injection events, and thereby obtaining a set of qualified groups of injection events, wherein each qualified group of injection events comprises a group-time indicator;
for each respective qualified group of injection events within the set of qualified groups of injection events:
   (i) determining, on a temporal basis, a subset of grouped medicament records (242) corresponding to the respective qualified group of injection events (241), using the group-time indicator (243) and the relative time of each of the medicament records (222) in each selected subset of medicament records (231) of the set of selected subsets of medicament records (230), and thereby obtaining a subset of grouped medicament records,
   (ii) processing the subset of grouped medicament records (242) of the respective qualified group of injection events to obtain display data (249) configured to represent a measure of central tendency and a measure of variability of injection events within the respective qualified group of injection events (241), wherein the measure of central tendency (244, 246) and the measure of variability (245, 247) is related to the relative time (237); and
   communicating the display data (249) to (i) the subject, (ii) to a health care provider, or (iii) to the user of the device (250), and thereby communicating the central tendency and the variability of the injection events.

In a further aspect is provided a computer program comprising instructions that, when executed by a computer having one or more processors and a memory, perform the method, as defined above.

In a further aspect is provided a computer-readable data carrier having stored thereon the computer program as defined above.

### BRIEF DESCRIPTION OF THE DRAWINGS

FIG. 1 illustrates an exemplary system topology that includes for communicating a dose history configured for representing a central tendency and a variability of a distribution of injections with a blood glucose regulating medicament applied by a subject with a treatment regimen, a data collection device for collecting patient data, one or more glucose sensors that measure glucose data from the subject, one or more injection devices that are used by the subject to inject blood glucose regulating medicaments in accordance with the treatment regimen, and one or more wearable life-style measurement devices, where the above-identified components are interconnected, optionally through a communications network, in accordance with an embodiment of the present disclosure.

FIGS. 2A and 2B illustrates collectively a device for communicating a dose history configured for representing a central tendency and a variability of a distribution of injections with a blood glucose regulating medicament applied by a subject with a treatment regimen in accordance with an embodiment of the present disclosure.

FIGS. 3A, 3B, 3C, 3D, 3E, 3F, 3G, 3I and 3J illustrate exemplary embodiments of devices for communicating a dose history configured for representing a central tendency and a variability of a distribution of injections with a blood glucose regulating medicament applied by a subject with a treatment regimen in accordance with another embodiment of the present disclosure.

FIG. 4 provide a flow chart of processes and features of a device for communicating a dose history configured for representing a central tendency and a variability of a distribution of injections with a blood glucose regulating medicament applied by a subject with a treatment regimen, in accordance with various embodiments of the present disclosure.

FIGS. 5A, 5B and 5C illustrates steps from the process provided in FIG. 4 and in accordance with an embodiment of the present disclosure.

FIGS. 6A, 6B and 6C illustrates further aspects of the process provided in FIG. 4 and in accordance with an embodiment of the present disclosure.

FIGS. 7A and 7B illustrates alternatives of communicated display data in accordance with an embodiment of the present disclosure.

FIGS. 8A and 8B illustrates further alternatives of communicated display data in accordance with an embodiment of the present disclosure.

FIGS. 9a and 9B collectively illustrates the effect of communicating a dose history configured for representing a central tendency and a variability of a distribution of injections with a blood glucose regulating medicament applied by a subject with a treatment regimen according to the invention. FIG. 9B illustrates a communicated display data in accordance with as aspect of the present invention, and FIG. 9A illustrates a communication of display data without grouping of injection events in order to represent a central tendency and a variability.

FIG. 10 illustrates an alternative of communicated display data in accordance with an embodiment of the present disclosure, where display data has been communicated in a table.

Like reference numerals refer to corresponding parts throughout the several views of the drawings.

### DETAILED DESCRIPTION

The present disclosure relies upon the acquisition of a data set comprising a plurality of blood glucose regulating medicament records taken over a time course. Each respective blood glucose regulating medicament record in the plurality of blood glucose regulating medicament records comprises (i) a respective blood glucose regulating medicament injection event including an amount of blood glucose regulating medicament injected into a subject using a respective injection device in a set of one or more injection devices, and (ii) a corresponding electronic injection event timestamp within the time course that is automatically generated by the respective injection device upon occurrence of the respective blood glucose regulating medicament injection event.

FIG. 1 illustrates an example of an integrated system 105 for the acquisition of such data. The integrated system 105 includes one or more connected injection devices 104, one or more glucose sensors 102, one or more wearable life-style measurement devices (103), memory (not shown), and a processor (not shown). In some embodiments, a glucose sensor 102 is a continuous glucose monitor. In some embodiments, a continuous glucose monitor will be able to timestamp a life-style event, e.g. meal ingestion or fasting period, which the subject engaged in, and therefore it can for this purpose be regarded as wearable a life-style measurement device.

With the integrated system 105, data from the one or more injection devices 104, used to apply a treatment regimen to the subject, is obtained as a plurality of insulin medicament records. Each insulin medicament record comprises a time stamped event specifying an amount of injected blood glucose regulating medicament that the subject received as part of the treatment regimen. Also, in some embodiments, autonomous time stamped glucose measurements of the subject are obtained. In such embodiments, the autonomous glucose measurements are filtered and stored in a non-transitory memory. The plurality of blood glucose regulating medicament records of the subject taken over a time course are used to provide a dose history for representing a central tendency and a variability of a distribution of the injections. In this way, the blood glucose medicament records are retrieved and communicated in accordance with the methods of the present disclosure.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein. By the term insulin pen is meant an injection device suitable for applying discrete doses of insulin, where the injection device is adapted for logging and communicating dose related data.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

A detailed description of a system 48, for communicating a dose history configured for representing a central tendency and a variability of a distribution of injections with a blood glucose regulating medicament in accordance with the present disclosure, is described in conjunction with FIGS. 1 through 3. As such, FIGS. 1 through 3 collectively illustrate the topology of the system in accordance with the present disclosure. In the topology, there is a dose history communication device (250) communicating injections performed by a subject who has applied a treatment regimen (206) within a time course (FIGS. 1, 2, and 3), a device for data collection (“data collection device 200”), one or more injection devices 104 for injecting medicaments into the subject, and optionally one or more glucose sensors 102 associated with the subject. Throughout the present disclosure, the data collection device 200 and the dose history communication device 250 will be referenced as separate devices solely for purposes of clarity. That is, the disclosed functionality of the data collection device 200 and the disclosed functionality of the dose history communication device 250 are contained in separate devices as illustrated in FIG. 1. However, it will be appreciated that, in fact, in some embodiments, the disclosed functionality of the data collection device 200 and the disclosed functionality of the dose history communication device 250 are contained in a single device. In some embodiments, the disclosed functionality of the data collection device 200 and/or the disclosed functionality of the dose history communication device 250 are contained in a single device and this single device is a smart phone. In some embodiments, the treatment regimen (206) comprises a bolus insulin medicament dosage regimen with a short acting insulin medicament or a basal insulin medicament dosage regimen with a long acting insulin medicament. In some embodiment the treatment regimen may also comprise a dosage regimen with a medicament comprising a GLP-1 receptor agonist as liraglutide or semaglutide.

Referring to FIG. 1, the dose history communication device 250 communicates a dose history configured for representing a central tendency and a variability of a distribution of injections applied by the subject. To do this, the data collection device 200, which is in electrical communication with the dose history communication device 250, receives a plurality of blood glucose regulating medicament records over a time course, each record comprising (i) a blood glucose regulating medicament injection event including an amount of blood glucose regulating medicament injected into the subject using a respective injection device 104 in the one or more injection devices, (ii) a corresponding electronic injection event timestamp that is generated by the respective injection device upon occurrence of the blood glucose regulating medicament injection event, and (iii) a respective type of blood glucose regulating medicament (if more than one medicament is applied) injected into the subject from one of short and long acting insulin medicament, and alternatively also a medicament comprising a GLP-1 receptor agonist. In some embodiments, the data collection device 200 also receives glucose measurements from one or more glucose sensors (e.g., continuous glucose monitors/sensors) 102 used by the subject to measure glucose levels. In some embodiments, the data collection device 200 receives such data directly from the injection devices 104 and/or glucose sensor(s) 102 and/or wearable life-style measurement device (103) used by the subject. For instance, in some embodiments, the data collection device 200 receives this data wirelessly through radio-frequency signals. In some embodiments, such signals are in accordance with an 802.11 (WiFi), Bluetooth, or ZigBee standard. In some embodiments, the data collection device 200 receives such data directly, analyzes the data, and passes the analyzed data to the dose history communication device 250. In some embodiments, an injection device 104, which can be an insulin pen, and/or a glucose sensor 102, and or wearable life-style measurement device (103) includes an RFID tag and communicates to the data collection device 200 and/or the dose history communication device 250 using RFID communication. In some embodiments, the data collection device 200 also receives life-style related event measurements from one or more wearable life-style measurement devices (e.g., meal ingestion sensor measuring a swallowing action, accelerometer measuring exercise etc.) (103) used by the subject to measure the occurrence of a life-style event, the beginning or the ending of such an event and/or to quantify how much the event may affect the blood glucose level of the subject. In some embodiments, the life style measurement device may also generate physiological measurements of the subject (e.g., from wearable physiological measurement devices, or from measurement devices within the data collection device 200 such as a thermometer, etc.).

In some embodiments, the data collection device 200 and/or the dose history communication device 250 is not proximate to the subject and/or does not have wireless capabilities or such wireless capabilities are not used for the purpose of acquiring medicament injection data, autonomous glucose data, and/or life-style related measurement data. In such embodiments, a communication network 106 may be used to communicate insulin medicament injection data from the one or more injection devices 104 to the data collection device 200 and/or the dose history communication device 250, and/or autonomous glucose measurements from the glucose sensor 102 to the data collection device 200 and/or the dose history communication device 250, and/or life-style related event data from one or more life-style measurement devices to the data collection device 200 and/or the dose history communication device 250.

Examples of networks 106 include, but are not limited to, the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of the present disclosure.

In some embodiments, the data collection device 200 and/or the dose history communication device 250 is part of an insulin pen. That is, in some embodiments, the data collection device 200 and/or the dose history communication device 250 and an injection device 104 are a single device.

In some embodiments, there is a single glucose sensor 102 attached to the subject and the data collection device 200 and/or the dose history communication device 250 is part of the glucose sensor 102. That is, in some embodiments, the data collection device 200 and/or the dose history communication device 250 and the glucose sensor 102 are a single device.

Of course, other topologies of the system 48 are possible. For instance, rather than relying on a communications network 106, the one or more injection devices 104 and the optional one or more glucose sensors 102 may wirelessly transmit information directly to the data collection device 200 and/or dose history communication device 250. Further, the data collection device 200 and/or the dose history communication device 250 may constitute a portable electronic device, a server computer, or in fact constitute several computers that are linked together in a network or be a virtual machine in a cloud computing context. As such, the exemplary topology shown in FIG. 1 merely serves to describe the features of an embodiment of the present disclosure in a manner that will be readily understood to one of skill in the art.

Referring to FIG. 2, in typical embodiments, the dose history communication device 250 comprises one or more computers. For purposes of illustration in FIG. 2, the dose history communication device 250 is represented as a single computer that includes all of the functionality for communicating a dose history for representing a central tendency and a variability of a distribution of injections with a blood glucose regulating medicament applied by a subject with a treatment regimen. However, the disclosure is not so limited. In some embodiments, the functionality for communicating the dose history is spread across any number of networked computers and/or resides on each of several networked computers and/or is hosted on one or more virtual machines at a remote location accessible across the communications network 106. One of skill in the art will appreciate that any of a wide array of different computer topologies are used for the application and all such topologies are within the scope of the present disclosure.

Turning to FIG. 2 with the foregoing in mind, an exemplary dose history communication device 250 for communicating a dose history for representing a central tendency and a variability of a distribution of injections with a blood glucose regulating medicament comprises one or more processing units (CPU's) 274, a network or other communications interface 284, a memory 192 (e.g., random access memory), one or more magnetic disk storage and/or persistent devices 290 optionally accessed by one or more controllers 288, one or more communication busses 213 for interconnecting the aforementioned components, a user interface 278, the user interface 278 including a display 282 and input 280 (e.g., keyboard, keypad, touch screen), and a power supply 276 for powering the aforementioned components. In some embodiments, data in memory 192 is seamlessly shared with non-volatile memory 290 using known computing techniques such as caching. In some embodiments, memory 192 and/or memory 290 includes mass storage that is remotely located with respect to the central processing unit(s) 274. In other words, some data stored in memory 192 and/or memory 290 may in fact be hosted on computers that are external to the dose history communication device 250 but that can be electronically accessed by the dose history communication device 250 over an Internet, intranet, or other form of network or electronic cable (illustrated as element 106 in FIG. 2) using network interface 284.

In some embodiments, the memory 192 of the dose history communication device 250 for communicating a dose history representing an average and a variability of a distribution of the injections applied by the subject stores:
an operating system 202 that includes procedures for handling various basic system services;
a medicament duration of action profile for the blood glucose regulating medicament that is characterized by a duration of the blood glucose regulating medicament (not shown on figure);
a dose history communication module 204;
a treatment regimen 206 which the subject is engaged in;
a first data set 220 from one or more injection devices used by the subject to apply the treatment regimen, the first data set comprising a plurality of medicament records over a time course, each respective medicament record 222 in the plurality of medicament records comprising: (i) a respective medicament injection event 224 including an amount of medicament 226 injected into the subject using a respective injection device 104 in the one or more injection devices, (ii) a corresponding electronic injection event timestamp 229 within the time course that is automatically generated by the respective injection device 104 upon occurrence of the respective medicament injection event;
a plurality of consecutive time windows 233 within the time course, wherein each time window 234 is of the same fixed duration,
for each respective time window 234, a subset of medicament records 235 comprising a number of medicament records 222, wherein this number can be zero if the subset of medicament records 235 is empty;
for each respective medicament record 222 within the respective subset of medicament records 235, a corresponding relative time 237 being the relative time within the time window;
a set of selected subsets of medicament records 230,
for each respective subset of medicament record 231, a medicament record 222 and a relative time 237;
a set of qualified groups of injection events 240,
for each group of qualified group of injection events 241, a subset of grouped medicament records 242, a group-time indicator 243, a medicament record 222, and in some further embodiments also a measure of central tendency of time 244, a measure of variability of time 245, a measure of central tendency of amount of injected blood glucose regulating medicament 246, and a measure of variability of the amount of blood glucose regulating medicament 247;
display data 247 comprising a measure of central tendency of time 244, a measure of variability of time 245, and in some further embodiments also a measure of central tendency of amount of injected blood glucose regulating medicament 246, and a measure of variability of the amount of blood glucose regulating medicament 247.

In some embodiments, the dose history communication module 204 is accessible within any browser (phone, tablet, laptop/desktop). In some embodiments the dose history communication module 204 runs on native device frameworks, and is available for download onto the dose history communication device 250 running an operating system 202 such as Android or iOS.

In some implementations, one or more of the above identified data elements or modules of the dose history communication device 250 for communicating a dose history configured for representing a central tendency and a variability of a distribution of injections are stored in one or more of the previously described memory devices, and correspond to a set of instructions for performing a function described above. The above-identified data, modules or programs (e.g., sets of instructions) need not be implemented as separate software programs, procedures or modules, and thus various subsets of these modules may be combined or otherwise re-arranged in various implementations. In some implementations, the memory 192 and/or 290 optionally stores a subset of the modules and data structures identified above. Furthermore, in some embodiments, the memory 192 and/or 290 stores additional modules and data structures not described above.

In some embodiments, a dose history communication device 250 for communicating a dose event history representing an average and a variablity of a distribution of injections is a smart phone (e.g., an iPHONE), laptop, tablet computer, desktop computer, or other form of electronic device (e.g., a gaming console). In some embodiments, the dose history communication device 250 is not mobile. In some embodiments, the dose history communication device 250 is mobile.

FIG. 3A to 3J provides collectively a further description of specific embodiments of a dose history communication device 250 that can be used with the instant disclosure. The dose history communication device 250 illustrated in FIGS. 3A to 3J has one or more processing units (CPU's) 274, peripherals interface 370, memory controller 368, a network or other communications interface 284, a memory 192 (e.g., random access memory), a user interface 278, the user interface 278 including a display 282 and input 280 (e.g., keyboard, keypad, touch screen), an optional accelerometer 317, an optional GPS 319, optional audio circuitry 372, an optional speaker 360, an optional microphone 362, one or more optional intensity sensors 364 for detecting intensity of contacts on the dose history communication device 250 (e.g., a touch-sensitive surface such as a touch-sensitive display system 282 of the dose history communication device 250), an optional input/output (I/O) subsystem 366, one or more optional optical sensors 373, one or more communication busses 213 for interconnecting the aforementioned components, and a power supply 276 for powering the aforementioned components.

In some embodiments, the input 280 is a touch-sensitive display, such as a touch-sensitive surface. In some embodiments, the user interface 278 includes one or more soft keyboard embodiments. The soft keyboard embodiments may include standard (QWERTY) and/or non-standard configurations of symbols on the displayed icons.

The dose history communication device 250 illustrated in FIGS. 3A to 3J optionally includes, in addition to accelerometer(s) 317, a magnetometer (not shown) and a GPS 319 (or GLONASS or other global navigation system) receiver for obtaining information concerning the location and orientation (e.g., portrait or landscape) of the dose history communication device 250 and/or for determining an amount of physical exertion by the subject.

It should be appreciated that the dose history communication device 250 illustrated in FIGS. 3A to 3J is only one example of a multifunction device that may be used for communicating a dose event history representing an average and a variability of a distribution of injections, and that the dose history communication device 250 optionally has more or fewer components than shown, optionally combines two or more components, or optionally has a different configuration or arrangement of the components. The various components shown in FIG. 3A are implemented in hardware, software, firmware, or a combination thereof, including one or more signal processing and/or application specific integrated circuits.

Memory 192 of the dose history communication device 250 illustrated in FIG. 3A to 3J optionally includes high-speed random access memory and optionally also includes non-volatile memory, such as one or more magnetic disk storage devices, flash memory devices, or other non-volatile solid-state memory devices. Access to memory 192 by other components of the dose history communication device 250, such as CPU(s) 274 is, optionally, controlled by the memory controller 368.

The peripherals interface 370 can be used to couple input and output peripherals of the device to CPU(s) 274 and memory 192. The one or more processors 274 run or execute various software programs and/or sets of instructions stored in memory 192, such as the dose history communication module 204, to perform various functions for the dose history communication device 250 and to process data.

In some embodiments, the peripherals interface 370, CPU(s) 274, and memory controller 368 are, optionally, implemented on a single chip. In some other embodiments, they are implemented on separate chips.

RF (radio frequency) circuitry of network interface 284 receives and sends RF signals, also called electromagnetic signals. In some embodiments, the standing treatment regimen 206, the first data set 220, and/or the second data set, and/or the third data set is received using this RF circuitry from one or more devices such as a glucose sensor 102 associated with a subject, an injection device 104 associated with the subject, a the life-style measurement device 103, and/or the data collection device 200. In some embodiments, the RF circuitry 284 converts electrical signals to/from electromagnetic signals and communicates with communications networks and other communications devices, glucose sensors 102, and injection devices 104 and/or the life-style measurement device 200 via the electromagnetic signals. The RF circuitry 284 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 284 optionally communicates with the communication network 106. In some embodiments, the circuitry 284 does not include RF circuitry and, in fact, is connected to the network 106 through one or more hard wires (e.g., an optical cable, a coaxial cable, or the like).

In some embodiments, the audio circuitry 372, the optional speaker 360, and the optional microphone 362 provide an audio interface between the subject and the dose history communication device 250. The audio circuitry 372 receives audio data from the peripherals interface 370, converts the audio data to electrical signals, and transmits the electrical signals to the speaker 360. The speaker 360 converts the electrical signals to human-audible sound waves. The audio circuitry 372 also receives electrical signals converted by the microphone 362 from sound waves. The audio circuitry 372 converts the electrical signal to audio data and transmits the audio data to peripherals interface 370 for processing. Audio data is, optionally, retrieved from and/or transmitted to the memory 192 and/or the RF circuitry 284 by the peripherals interface 370.

In some embodiments, the power supply 276 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

In some embodiments, the dose history communication device 250 optionally also includes one or more optical sensors 373. The optical sensor(s) 373 optionally include charge-coupled device (CCD) or complementary metal-oxide semiconductor (CMOS) phototransistors. The optical sensor(s) 373 receive light from the environment, projected through one or more lenses, and converts the light to data representing an image. The optical sensor(s) 373 optionally capture still images and/or video. In some embodiments, an optical sensor is located on the back of the dose history communication device 250, opposite the display 282 on the front of the dose history communication device 250, so that the input 280 is enabled for use as a viewfinder for still and/or video image acquisition. In some embodiments, another optical sensor 373 is located on the front of the dose history communication device 250 so that the subject's image is obtained (e.g., to verify the health or condition of the subject, to determine the physical activity level of the subject, to help diagnose a subject's condition remotely, or to acquire visual physiological measurements of the subject, etc.).

As illustrated in FIG. 3A to 3J, a dose history communication device 250 preferably comprises an operating system 202 that includes procedures for handling various basic system services. The operating system 202 (e.g., iOS, DARWIN, RTXC, LINUX, UNIX, OS X, WINDOWS, or an embedded operating system such as VxWorks) includes various software components and/or drivers for controlling and managing general system tasks (e.g., memory management, storage device control, power management, etc.) and facilitates communication between various hardware and software components.

In some embodiments the dose history communication device 250 is a smart phone. In other embodiments, the dose history communication device 250 is not a smart phone but rather is a tablet computer, desktop computer, emergency vehicle computer, or other form or wired or wireless networked device. In some embodiments, the dose history communication device 250 has any or all of the circuitry, hardware components, and software components found in the dose history communication device 250 depicted in FIG. 2 or 3. In the interest of brevity and clarity, only a few of the possible components of the dose history communication device 250 are shown in order to better emphasize the additional software modules that are installed on the dose history communication device 250.

While the system 48 for communicating a dose history for communicating a dose history configured for representing a central tendency and a variability of a distribution of injections disclosed in FIG. 1 can work standalone, in some embodiments it can also be linked with electronic medical records to exchange information in any way.

As illustrated in FIG. 3A to 3J, in some embodiments, the memory 192 of the dose history communication device 250 for communicating a dose history representing an average and a variability of a distribution of the injections applied by the subject further stores one or more of the following data structures.

FIG. 3A illustrates an example of an embodiment further storing a probability density function 310 of the distribution of injection events, and a set of identified groups of injection events 320.

FIG. 3B illustrates another example of an embodiment where the memory 192 further comprises a probability density function 310, a set of identified groups of injection events, wherein each identified group of injection events 321 comprises a Peak 322 having a peak value 323 and a peak time being the group-time indicator. For the exemplary embodiment the memory further stores a pre-defined threshold for evaluating whether or not an identified group of injections 321 is qualified to be communicated.

FIG. 3C illustrates another exemplary embodiment where the memory further comprises a set of pre-defined time-ranges 330 indicating a time range of interest for evaluation. Each pre-defined time-range 331 is a group-time indicator, and the pre-defined time range is associated with a subset of identified medicament records 332, and a number of medicament records 333 being the number of medicament records in the subset of medicament records 332, and an identified group of injection events 321. The memory 192 further stores a pre-defined threshold for evaluating whether or not an identified group of injections 321 is qualified to be communicated.

FIG. 3D illustrates an alternative exemplary embodiment where the memory 192 further stores a user input 327 threshold for evaluating whether or not an identified group of injections 321 is qualified to be communicated.

FIG. 3E illustrates an alternative exemplary embodiment where the memory 192 further stores a shape data structure 350 for structuring data configured to be graphically displayed, the shape data structure 350 comprises a central tendency data structure 351 for structuring a central tendency polygon 352 configured for graphically illustrating a two-dimensional polygon visually indicating a position of the central tendency of the relative time. The data structure similarly comprises a variability data structure 353 structuring a variability polygon 349 for visually indicating the variability of the relative time. The central tendency polygon 352 and the variability polygon 349 are corresponding to the same variable, and they may comprise further data for illustrating the central tendency of other variables as the magnitude of injected medicament, or the relative time of a life-style event.

FIG. 3F illustrates an alternative exemplary embodiment where the memory 192 further stores a table data structure 354 structuring data to be displayed in a table, the data structure 354 comprises as an example a qualified group identification 355, a time variation 356, a median dose 358 and a dose variation 359. Alternatively, a table data structure could structure data for communicating a mean and a standard deviation.

FIG. 3G illustrates an alternative exemplary embodiment where the memory 192 further stores a frequency indicator 334 for indicating the frequency of injections for a corresponding qualified group of injections, the frequency indicator can for example specify a transparency value for displaying a polygon with higher transparency if the frequency of injections is relatively low, where the polygon is a two-dimensional filled structure for illustrating the central tendency or the variability.

FIG. 3H illustrates an alternative exemplary embodiment where the memory 192 further stores a normalized probability density function 311 a normalized peak value 325 for indicating the frequency of injections for a corresponding qualified group of injections, the normalized peak value can for example specify a transparency value for displaying polygon with higher transparency if the frequency of injections is relatively low, where the polygon is a two-dimensional filled structure for illustrating the central tendency or the variability.

FIG. 3I illustrates an alternative exemplary embodiment where the memory 192 further stores for each medicament record a type of medicament record, which for example could be fast acting insulin, long acting insulin or a medicament comprising a GLP-1 like protein.

FIG. 3J illustrates an alternative exemplary embodiment where the memory 192 further stores a second data set 340 comprising a plurality of autonomous glucose measurements within the time course, and wherein each autonomous glucose measurement 341 is associated with a glucose measurement time stamp 342.

In embodiments where autonomous glucose measurements are used, devices such as the FREESTYLE LIBRE CGM by ABBOTT (“LIBRE”) may serve as the glucose sensor 102 in order to make the plurality of autonomous glucose measurements of a subject. The LIBRE allows calibration-free glucose measurements with an on-skin coin-sized sensor, which can send up to eight hours of data to a reader device (e.g., the data collection device 200 and/or the dose history communication device 250) via near field communications, when brought close together. The LIBRE can be worn for fourteen days in all daily life activities. In some embodiments, the autonomous glucose measurements are autonomously taken from the subject at an interval rate of 5 minutes or less, 3 minutes or less, or 1 minute or less. In some embodiments, the autonomous glucose measurements are taken from the subject at an interval rate of 5 minutes or less, 3 minutes or less, or 1 minute or less, over a time period of a day or more, two days or more, a week or more, or two weeks or more. In some embodiments, the autonomous glucose measurements are autonomously taken (e.g., without human effort, without human intervention, etc.).

Now that details of a system 48 for for communicating a dose event history for communicating a dose history configured for representing a central tendency and a variability of a distribution of injections have been disclosed, details regarding a flow chart of processes and features of the system, in accordance with an embodiment of the present disclosure, are disclosed with reference to FIG. 4. In some embodiments, such processes and features of the system are carried out by the insulin dosage adjustment module 204 illustrated in FIGS. 2 and 3.

Blocks 402-422. With reference to FIG. 4, the goal of insulin therapy in subjects with either type 1 diabetes mellitus or type 2 diabetes mellitus is to match as closely as possible normal physiologic insulin secretion to control fasting and postprandial plasma glucose, and data collection and presentation is an important component in order to understand the progress in the treatment. As illustrated in FIG. 2, a device 250 is provided for communicating a dose history configured for communicating a dose history configured for representing a central tendency and a variability of a distribution of injections with a blood glucose regulating medicament applied by a subject with a treatment regimen. The device comprises one or more processors 274 and a memory 192/290, the memory storing instructions that, when executed by the one or more processors, performs a method which will be described below and illustrated in FIG. 4. By configuring the processor, the memory and the stored instructions, as described above, the dose history communication device 250 is configured or adapted to perform the method.

Referring to FIG. 4, the block 402 indicates a starting point of the method, and block 404 represent a step of obtaining a first data set 220 from one or more injection devices 104 used by the subject to apply the treatment regimen 206. The first data set 220 comprises a plurality of medicament records taken over a time course, each respective medicament record 222 in the plurality of medicament records comprises: (i) a respective medicament injection event 224 including an amount of medicament 226 injected into the subject using a respective injection device 104 in the one or more injection devices, (ii) a corresponding electronic injection event timestamp 229 within the time course that is automatically generated by the respective injection device upon occurrence of the respective medicament injection event 224.

Block 406 represents another step of the method, wherein the step comprises creating a plurality of consecutive time windows 233 within the time course, wherein each time window 234 is of the same fixed duration, as illustrated on the upper part of FIG. 5A.

For each respective time window 234, another step, represented by block 408, comprises creating a subset of medicament records 235, and thereby implicitly creating a plurality of sets of medicament records 533, as also illustrated on FIG. 5A. In this way, each respective subset of medicament records 235 in the plurality of medicament records 533, comprises a number of medicament records from the first data set 220, and each respective medicament injection event 224 or medicament record 222 within the respective subset of medicament records 235 have a timestamp 229 in the respective time window 234.

Block 410 represents another step of the method, also illustrated on FIG. 5A. For each respective medicament record 222, within each subset of medicament records 235 of the plurality of sets of medicament records 533, the step comprises assigning a corresponding relative time 237 to the respective medicament record 222. For this purpose, the relative time is defined as the relative time within the window 234, e.g., measured as the time from the beginning of the time window to the point in time in the time window indicating the incidence of the injection event. The incidence of the injection in the time window is identified by the time stamp. In this way, the plurality of sets of medicament records 533 represents the distribution of injections.

Block 412 represents another step of the method illustrated in FIG. 5B. The method further comprises selecting a set of subsets of medicament records 235 from the plurality of subsets of medicament records 533 and thereby obtaining a set of selected subsets of medicament records 230 comprising a number of selected subsets of medicament records 231 representing a distribution of injection events within an interval corresponding to the fixed duration of the time windows 234. The distribution is illustrated in the window W1 on FIG. 5B, and the arrows A1 to A2 illustrates how the medicament records of one of the selected subsets of medicament records 231 is distributed in the distribution.

Block 414 represents another step of the method illustrated in FIG. 5C. The method further comprises obtaining one or more qualified groups of injection events within the distribution of injection events, and thereby obtaining a set of qualified groups of injection events 240, wherein each qualified group of injection events comprises a group-time indicator 243. The distribution is illustrated in the window W2 on FIG. 5C, and the qualified groups are indicated by dashed rectangles QG1 and QG2.

Block 416 represents another step of the method illustrated in FIG. 5C. The method further comprises, for each respective qualified group of injection events 241 within the set of qualified groups of injection events 240: determining, on a temporal basis, a subset of grouped medicament records 242 corresponding to the respective qualified group of injection events 241, using the group-time indicator 243 and the relative time 237 of each of the medicament records 222 in each selected subset of medicament records 231 of the set of selected subsets of medicament records 230, and thereby obtaining a subset of grouped medicament records 242.

Block 418 represents another step of the method illustrated in FIG. 5C. The method further comprises, for each respective qualified group of injection events 241 within the set of qualified groups of injection events 240: processing the subset of grouped medicament records 242 of the respective qualified group of injection events to obtain display data 249 configured to represent a measure of central tendency and a measure of variability of injection events within the respective qualified group of injection events 241, wherein the measure of central tendency 244, 246 and the measure of variability 245, 247 is related to the relative time 237.

Block 420 represents another step of the method further comprising communicating the display data 249 to (i) the subject, (ii) to a health care provider, or (iii) to the user of the device 250, and thereby communicating the central tendency and the variability of the injection events. Block 422 illustrates the end of the process.

In a further aspect, the step of processing the subset of grouped medicament records 242 of the respective qualified group of injection events to obtain display data 249, represented in block 418, can further comprise processing the subset of grouped medicament records 242 of the respective qualified group of injection events to obtain display data 249 configured to represent a measure of central tendency and a measure of variability of injection events within the respective qualified group of injection events (241), wherein the measure of central tendency (244, 246) and the measure of variability (245, 247) is related to the amount of medicament (226) injected into the body.

FIG. 6A to 6E illustrates further aspects of exemplary embodiments of the disclosure, and of the analysis of insulin injection dosing by grouping of injection events. Insulin dose data from a period of days is loaded into a computer system (PC, web-based platform, mobile phone, etc). Insulin doses can be from one or more pen injection devices 104, containing one or more different medicines, i.e. long-acting and rapid insulin. To get the full benefit of this solution, the system also has glucose data from the same period. Data regarding meals or exercise can also be a part of this system.

Analysis of the multiple injection pens 104 is done separately. The following description is the generic analysis for one pen. Visualisation of the resulting analysis may combine both pens to be viewed together, or may be separate.

In a further aspect, a further step of the process within block 414 is to determine or identify groups of injection events in a distribution, based on the time at which the dosing occurred, i.e., on a temporal basis. In other words, the step, represented in block 414, of obtaining one or more qualified groups of injection events within the distribution of injection events, and thereby obtaining a set of qualified groups of injection 240 events further comprises:
estimating a probability density function 310 of the distribution of injections events, by using the set of selected subsets of medicament records 230;
identifying one or more groups of injection events within the distribution of injection events, by using the probability density function 310, wherein each of the identified groups of injection events 321 are identified by a peak 322 indicating a local maximum of the probability density function 310, wherein the identified peak comprises a peak value 323 and a corresponding peak time 324, and thereby obtaining a set of identified groups of injection events 320;
responsive to identifying one or more groups of injection events, for each respective identified group of injection events 321 within the set of identified groups of injection events 320, evaluating whether the respective identified group of injection events 321 is qualified to be communicated, and thereby obtaining a set of qualified groups of injection events 240; and
wherein the step of determining the subset of grouped medicament records 242, represented by block 416, for each respective qualified group of injection events 241 further comprises using the peak-time 324 as the group-time indicator 243.

In a further aspect, the above mentioned step, of evaluating whether the respective identified group of injection events is qualified to be communicated, further comprises evaluating whether a function of the peak value 323 satisfies a pre-defined threshold for qualification 329.

FIG. 6A illustrates a distribution of medicament injection events, the individual medicament injection events are illustrated by circles, and are in this example illustrating injections with a rapid acting insulin. FIG. 6A further illustrates the identification process by using a Kernel density estimation, which is a way to estimate the probability density function of a random variable. A data set, e.g. the set of selected subsets of medicament records 230, is created including the points in time when an injection occurred. For example, if the data being examined is for rapid insulin injections, the data set is called: Time_rapid. The graphical depiction is shown as the circles in FIG. 6A.

Then, using the previously defined algorithm for kernel density estimation, the density function is determined:

KDF_rapid=f(Time_rapid)

For this example, we used a Kernel density estimator provided by mathworks, www.mathworks.com/matlabcentral/fileexchange/14034-kernel-density-estimator

KDF_rapid is then used to generate a data set from 0 to 24 hours, which is an example of a time window 234 with a fixed width of 24 hours. The data set Density_rapid is representing the probability density, i.e. a probability density function 310, of rapid injections:

Density_rapid=f(Time_0to24 hours,KDF_rapid)

For simplification later, this data is normalized based on the max Density, so all values are between 0 and 1. The normalized probaility function 311, exemplified as Density_rapid is illustrated on FIG. 6A as a solid line for explanation purposes.

The probability density function 310 is then used to determine when a group of injection events is qualified. One method of doing this is to first identify the approximate midpoint of a group, the peak time 326, based on a peak 325 of the normalized density function 311. Taking the numerical derivative of Density_rapid (shown as dashed line on FIG. 6A), and then finding where the derivative is equal to zero (first zero value is indicated by Z1) is one way of finding peaks. Some smaller peaks may exist, which are the results of a single or few injection points. Therefore, it may be useful to set a minimum, i.e. a pre-defined threshold for qalification 329, for the evaluation of peaks. In this example, a minimum was set to 0.15, so any peaks with a density value less than 0.15 are ignored, and are qualified for being displayed. This value could be set automatically, by the user, or adjusted to see which is most suitable for a given data set. The result of this analysis gives the following depiction which shows peaks at times 326 (07:28, 12:26, 16:43, 18:47).

With the peak times now representing approximate times of the different standard injection groups, it is then necessary to determine which injection doses belong to the groups. One method of doing this is to simply calculate the difference in time from each injection time to the various group times. The minimum of these time differences for each point then determines which group the injection belongs to. After this analysis, the plot in FIG. 6B shows the points in the various qualified groups of injection events. The members of a first qualified group 341-1 is represented by circles, the members of a second qualified group 341-2 is represented by plus symbols, the members of a third qualified group (341-3) is represented by asterisk symbols and the members of a fourth qualified group (321-4) is represented by diamond symbols. The members of the first qualified group of injection event 241-1 define a first subset of grouped medicament records 242-1, the members of the second qualified group of injection event 241-2 define a second subset of grouped medicament records 242-2 and so forth.

In another aspect, user-defined time ranges may be used for identifying injection event grouping as an alternative to using the probability density function. In this aspect, the step of obtaining one or more qualified groups of injection events within the distribution of injection events, represented by block 414, and thereby obtaining a set of qualified groups of injection events 240, further comprises:
obtaining a set of pre-defined time ranges 330, wherein each of the pre-defined time ranges 331 within the set of pre-defined time ranges 330 are defined within the fixed duration of the time windows, and wherein none of the pre-defined time ranges 331 are overlapping another pre-defined time range 331 within the set of pre-defined time ranges 330;
for each pre-defined time range 331, the method comprises:
obtaining an identified group of injection events 321 and thereby obtaining a set of identified groups of injection events 320,
evaluating whether the identified group of injection events 321 is a qualified group of injection events 241, and thereby obtaining a set of qualified groups of injection events 240; and
wherein the step of determining the subset of grouped medicament records 242, represented by block 416, for each respective qualified group of injections events 241, further comprises using the pre-defined time range 331 as the group-time indicator 243.

In a further aspect the number of medicament records within the subset of identified medicament records can be used for qualifying or disqualifying the identified subset. For each pre-defined time range 331, the above mentioned step of obtaining an identified group of injection events 321 further comprises:
determining, on a temporal basis, a subset of identified medicament records 332 of the medicament records 222 within the set of selected subsets of medicament records 230, wherein the medicament records 222 within the subset of identified medicament records 332 have a relative time 237 within the respective pre-defined time range 331; and
wherein the above mentioned step of evaluating whether the identified group of injection events 321 is a qualified group of injection events 241 further comprises:
evaluating the number of medicament records within the subset of identified medicament records 333,
evaluating whether the number of medicament records 333 is above a pre-defined threshold 329, wherein the identified group 221 is qualified, or below a pre-defined threshold 329, wherein the identified group (221) is disqualified;
responsive to the identified group 221 is qualified characterizing the identified group of injection events as a qualified group of injection events 241.

In a further aspect a user-input can enforce that an identified group is also a qualified group. For each pre-defined time range 331, the step mentioned above of evaluating whether the identified group of injection events 321 is a qualified group of injection events 241 further comprises:
obtaining a user input 322 indicating whether the pre-defined time range 331 is qualified or disqualified; and
responsive to the obtained user input indicates that the pre-defined time range 331 is qualified, characterizing the identified group of injection events as a qualified group of injection events.

In a further aspect a pre-defined input can enforce that an identified group is also a qualified group. For each pre-defined time range 331, the above mentioned step of evaluating whether the identified group of injection events 321 is a qualified group of injection events 241 further comprises:
obtaining a pre-defined input indicating whether the pre-defined time range (331) is qualified or disqualified; and
responsive to the obtained pre-defined input indicates that the pre-defined time range (331) is qualified, characterizing the identified group of injection events as a qualified group of injection events.

In other words, it can also be useful to permit the user (patient or HCP) to define time ranges which define injection event groups, rather than the automatic grouping defined above. In this case, the user could describe the group by name, start time and end time. For example, it could be that the user defines “lunch injection” as between 11:00 and 13:00. Then, all rapid insulin injections occurring in this time range would be included in this group. Similarly, the user could define “morning injection” as between 07:00 and 10:00. Then, all long-acting insulin injections occurring in this time range would be included in this group.

In a further aspect, the step of processing the subset of grouped medicament records, for each respective qualified group of injection events, to obtain display data configured to represent a central tendency and a variability of injection events, represented by block 418, comprises: evaluating a measure of central tendency for the relative time (237) and the amount of medicament (226) injected into the body, and evaluating a measure of variability for the relative time (237) and the amount of medicament (226) injected into the body.

In a further aspect, the step of determining a measure of central tendency comprises evaluating a median, and determining a measure of variability comprises evaluating an upper and a lower percentile.

In another aspect, the step of determining a measure of central tendency comprises evaluating a mean, and determining a measure of variability comprises evaluating a standard deviation.

Explained in more details, an analysis of injection data within each subset of grouped medicament records 242 follows. The grouped medicament records 242 are in the following denoted (G1, G2, . . . , Gn) for explanation purposes. First, the median time and median dose size are calculated and stored. The mean (average) dose could be substituted for median.

Median_time_Gn=median(Time_rapid_Gn)

Median_dose_Gn=median(Dose_rapid_Gn)

These values are stored as x,y pairs.

### (Median_time_G1, Median_dose_G1)

### (Median_time_G2, Median_dose_G2)

. . .

### (Median_time_Gn, Median_dose_Gn)

Then, the following percentile data are calculated for each group, and stored:

Injection times:

Percentile10_Time_Gn=percentile(10^{th},Time_rapid_Gn)

Percentile90_Time_Gn=percentile(90^{th},Time_rapid_Gn)

Percentile25_Time_Gn=percentile(25^{th},Time_rapid_Gn)

Percentile75_Time_Gn=percentile(75^{th},Time_rapid_Gn)

Injection doses:

Percentile10_Dose_Gn=percentile(10^{th},Dose_rapid_Gn)

Percentile90_Dose_Gn=percentile(90^{th},Dose_rapid_Gn)

Percentile25_Dose_Gn=percentile(25^{th},Dose_rapid_Gn)

Percentile75_Dose_Gn=percentile(75^{th},Dose_rapid_Gn)

Where the function percentile(p,data) calculates the pth percentile value for the data set data. In this example, the 10^{th}, 25^{th}, 75^{th}, and 90^{th} percentile values are calculated.

This completes the data analysis portion, and the next step is to generate plots for data visualisation, which is illustrated in FIG. 6C.

Therefore, in a further aspect, the step of processing the subset of grouped medicament records 242, for each respective qualified group of injection events 241, to obtain display data 249, represented by block 418, further comprises:
associating with each qualified group of injection events 241:
   a shape data structure 350, configured for representing the central tendency and the variability of the subset of grouped medicament records 242 corresponding to the respective qualified group of injection events 241, wherein the shape data structure 350 comprises:
      a central tendency data-structure 351 comprising a central tendency polygon 352 configured for visualizing a polygon with a two-dimensional shape indicating the measure of central tendency,
      a variability data-structure 353 comprising a variability polygon 354 configured for visualizing a polygon with a two-dimensional shape identifying the measure of variability.

The same procedure takes place for each group within the set of groups (G1, G2, . . . Gn). First, a hatched geometric shape 353-1 illustrated as a rectangle is generated based on the 10^{th} and 90th percentile values calculated, with relative time 357 on the x-axis and dose 226 on y-axis. This represents a region in which 80 percent (90 percent minus 10 percent) of all doses happened, for each group.

Next, a second shaded geometric shape 353-2, now with a more intense hatching, is generated based on the 25^{th} and 75^{th} percentile values. This represents a region in which 50 percent (75 percent minus 25 percent) of all doses happened, for each group.

Finally, a dark point 352 is constructed based on the median time and dose value, and FIG. 6C illustrates a graph zooming in to show the details of just one subset of grouped injections, and this will be referred to as the representation of a standard injection, representing injections obtained in a selected set of time windows.

The display data 249 obtained in the step represented by block 418 may comprise a variety of the obtained data structures. In one aspect, the display data comprises the data configured to represent an average and a variability of injection events within the respective qualified group of injection events. In a further aspect, the display data comprises the set of qualified groups of injection events and all the associated data structures and data.

In a further aspect, the method enables an indication of the frequency of injections in the respective qualified group of injection events. This is obtained by the step of processing the subset of grouped medicament records 242, for each respective qualified group of injection events 241, to obtain display data 249, represented in block 418, further comprises:
obtaining display data 249 configured to represent a frequency indicator 334 indicating the frequency of an injection event of the respective qualified group of injection events 241, wherein the frequency indicator 334 is a function of:
   the number of medicament records in the subset of grouped medicament records 242 over (i.e. divided by) the total number of medicament records in all subsets of grouped medicament records in the set of qualified groups of injection events 240, or
   the number of medicament records in the subset of grouped medicament records 242 over (i.e. divided by) the total number of medicament records of all selected subsets medicament records 231 in the set of selected subsets of medicament records 230;
thereby enabling an indication of the frequency of injections in the respective qualified group of injection events 242, and wherein the display data 249 further comprises the frequency indicator 334, which will eventually enable that the frequency indicator can be displayed.

In a further aspect the method enables an indication of the frequency of injections in the respective qualified group of injection events using the normalized probability distribution. This is obtained by the step of processing the subset of grouped medicament records 242, for each respective qualified group of injection events 241, to obtain display data 249, further comprises:
obtaining display data 249 configured to indicate a frequency of an injection event of the respective qualified group of injection events 241;
normalizing the probability density function 310 and the peak value to obtain a normalized peak value 325, whereby the normalized peak value 325 indicates the frequency of an injection event of the respective qualified group of injection events 241, and wherein the display data 249 comprises the normalized peak value 325.

In other words, one additional element of the invention is the possibility of changing the shading or any other continuous indicator, of the representation of the standard injection based on the frequency of injections at that time, compared to other standard injections or compared to a total number of injections. This can be done by utilizing the aforementioned normalized probability density value, which represents the likelihood of an injection occurring at that time. The intensity of the shading can be altered for each standard injection group, to reflect the relative density value. The graph of FIG. 7A show an example of this, where the standard injection just before 18:00, represented by the qualified group of injection 241-3, receives a lower intensity, and the other three (241-1, 241-2, 241-4) maintain high intensity, as a result of the density values: [0.97, 0.99, 0.24, 0.88]. The qualified group of injections 241-1′ represent injections from a distribution relating to basal injections, applied with a different pen, and an analysis of these data is obtained with a similar algorithm, but the analysis is executed separate from the analysis of rapid, fast, GLP-1 receptor agonist or ultra-fast injection data. In other words, the same process would take place for one or more injection pens, and these data would be plotted on the same graph or on a separate graph.

Then, this data is presented with blood glucose data, and possibly meal data and exercise data. FIG. 7A graph shows blood glucose data presented in the AGP format, along with standard day insulin doses from two types of insulin.

In a further aspect of the method the collected data can be handled according to the type of medicament to which they relate. In this further aspect, for each respective medicament record 222 in the plurality of medicament records, the method further comprises: a corresponding type of medicament 228 injected into the subject, and the set of selected subsets of medicament records all comprises the same type of medicament 228 and thereby represents a distribution of injection events corresponding to the respective type of medicament. The step of processing the subset of grouped medicament records 242, for each respective qualified group of injection events 241, to obtain display data 249 (represented by block 418), further comprises obtaining display data 249 configured to represent the respective type of medicament 228.

In a further aspect the treatment regimen comprises both a bolus insulin medicament dosage regimen with a short acting insulin medicament and a basal insulin medicament dosage regimen with a long acting insulin medicament, but data obtained on each of the types of medicament are handled separately eventhough they can be communicated in the same coordinate system or display.

In a further aspect the display data can be presented in a coordinate system, wherein the device further comprises a display 282 configured for representing a first coordinate system 710, as shown on FIGS. 7, 8 and 9B. In this further aspect, the step of communicating display data further comprises displaying the obtained display data 249, configured to represent a central tendency and a variability of injection events within the respective qualified group of injection events, in the first coordinate system 710 on the display 282, the first coordinate system comprises a first axis 711 and a second axis 712. The coordinate system may also comprise a third axis 713. The first coordinate system is adapted to represent the central tendency and the variability of the injection events. The first axis represents the relative time 237 and is defined within the interval defined by the time window 234, and the second axis 712 represents the amount of injected medicament 226.

In the case that the coordinate system comprises a third axis 713, the second axis 712 can relate to a first type of medicament and the third axis 713 can relate to a second type of medicament.

In a further aspect the device 250 may obtain both injection data and blood glucose data. In this further aspect method further comprises: obtaining a second data set 340, wherein the second data set 340 comprises a plurality of autonomous glucose measurements of the subject within the time course and, for each respective autonomous glucose measurement 341 in the plurality of autonomous glucose measurements, a glucose measurement timestamp 342 representing when the respective measurement was made. In this further aspect, the method also comprises, for each respective time window 234, creating a set of glucose measurements 345, and thereby creating a plurality of sets of glucose measurements, and wherein each glucose measurement 341 within the respective set of glucose measurements 345 have a timestamp 342 in the respective time window 234. For each respective glucose measurement 311, a corresponding relative time 343 being the relative time within the time window is associated, whereby the plurality of sets of glucose measurements are representing a distribution of glucose measurements within the time window. The method further comprises calculating, for the plurality of sets of glucose measurements, the central tendency and the variability as a function of the relative time, wherein the display data further comprises the plurality of sets of glucose measurements, the corresponding relative time, and the calculated central tendency and the variability as a function of the relative time.

In a further aspect injection data and glucose data are presented in a common display comprising two coordinate systems. In this further aspect, the device comprises a display 282 adapted to represent a first 710 and a second coordinate system 720 each comprising a first axis 711, 721 and a second axis 712, 722, and wherein the first coordinate system 710 is adapted to represent the central tendency and the variability of the injection events. The second coordinate system is adapted to represent the central tendency and the variability of the glucose data, and the step of communicating display data further comprises displaying the obtained display data 249, configured to represent an central tendency and a variability of injection events within a respective qualified group of injection events, in the first coordinate system 710 on the display 282. The method further comprises displaying the obtained display data 249, comprising an central tendency and a variability of the plurality of sets of glucose measurements as a function of time, in the second coordinate system 720 on the display 282. For the first coordinate system 710, the second axis 712 represents the amount of injected medicament 226, and, for the second coordinate system 720, the second axis 722 represents a blood glucose concentration. The first axis 711, 721 of each coordinate systems 710, 720 represent the relative time 237, 343 and are defined within the interval defined by the time window 234. The obtain the best opportunity for comparing injection data and blood glucose data the two coordinate systems should be synchronized, as shown on FIGS. 7-8.

FIG. 7-8 illustrates alternative display methods for illustrating the variability. FIG. 7A shows for each qualified group of injections a rectangle enabling the illustration of a lower and an upper limit in two dimensions.

FIG. 7B shows for each qualified group of injections an ellipse enabling the illustration of a lower and an upper limit in two dimensions.

FIG. 7C shows for each qualified group of injections a rectangle enabling the illustration of a lower and an upper limit in the time-dimension, and an upper limit in the dose-dimension. The lower limit of variation for the dose-dimension is not illustrated as the rectangle sets of at the base line.

FIG. 7D shows for each qualified group of injections a rectangle with rounded corners in one end and 90 degrees angle at the base line, the illustration enables the illustration of a lower and an upper limit in the time-dimension, and an upper limit in the dose-dimension. The lower limit of variation for the dose-dimension is not illustrated as the rectangle sets of at the base line.

A major advantage of an aspect of the disclosed invention is the ability to see how, on an average day, a patient injects insulin over a period of time, as well as the variability of time (and possibly also dose) with which a patient injects insulin. The grouping of injection events and visualisation of injection doses gives an HCP a quick summary of how the patient has, in a standard day, applied insulin injections in a way that visually compliments the way that AGP gives a picture of the standard day of blood glucose.

An example of this is shown in FIGS. 9A and 9B, which compares the visualisation of raw injection data (FIG. 9A) compared to injection data that has been automatically grouped and displayed via the median and percentile method (FIG. 9B). In this example, the patient is taking both rapid and long-acting insulin. The patient tends to take injections somewhat irregularly, causing the graph of raw data to appear somewhat random, without apparent groups.

In a further aspect the method is adapted for communicating display data in a table, as illustrated in FIG. 10. In this aspect, the step of processing the subset of grouped medicament records 242, for each respective qualified group of injection events 241, to obtain display data 249, represented by block 218, further comprises: associating with each qualified group of injection events 241 a table data structure 354 comprising: (i) a qualified group identification 355, (ii) a median time 356, and (iii) a lower and upper time variation 357 based on the relative time 237, of the subset of grouped medicament records 242, and (iv) a median dose 358 and a (v) dose variation 359 based on the amount of injected medicament 226 of the subset of grouped medicament records 242.

In other words, the obtained display data could also be included in the table of reported values shown above, as well as a difference between the recommended dose and median injected dose.

In a further aspect median points can be connected with a line or smoothed curve, to guide the eye, or the probability density curve or a modified version of it could be displayed along with injection data. In a further aspect, the analysis can be performed for different time periods (7 days, 2 weeks, 30 days, 90 days, etc), possibly in connection to insulin titration, where dose should be adjusted at defined intervals. The analysis can as an example also also be performed for Mondays only, Tuesdays only etc. In this case the selected subsets of medicament records correspond to the desired weekday. In a further aspect the subsets are selected with a desired periodicity, i.e., every Tuesday, every second Tuesday and so forth.

In a further aspect, the device is further adapted for communicating a life-style event history representing an central tendency and a variability of a distribution of life-style related events within the time course, which the subject has engaged in, wherein the method further comprises obtaining a third data set from one or more wearable life-style measurement devices 103 used by the subject to acquire life-style data, the third data set comprises a plurality of life-style data records over the time course, each respective life-style data record in the plurality of life-style data records comprises: (i) a respective life-style event including a measure of intensity indicating the effect on the subject using the respective measurement device (103), (ii) a corresponding electronic life-style event timestamp within the time course that is automatically generated by the respective life-style measurement device 103 upon occurrence of the respective life-style related event, or by user actuation of the respective life-style measurement device, or a begin timestamp and an end timestamp indicating the beginning and the ending time of the life-style event engaged in by the subject. The method further comprises, for each respective time window 234, obtaining a subset of life-style data records, and thereby obtaining a plurality of subsets of life-style data records, wherein each respective subset of life-style data records comprises a number of life-style data records from the third data set, and wherein each respective life-style data record within the respective subset of life-style data records have a timestamp in the respective time window 234. The method further comprises, for each respective life-style data record, within each subset of life-style data records of the plurality of subsets of life-style data records, assigning a corresponding relative time 237 being the relative time within the time window 234. The method further comprises, selecting a set of subsets of life-style data records from the plurality of subsets of life-style data records and thereby obtaining a set of selected subsets of life-style data records comprising a number of selected subsets of life-style data records representing a distribution of life-style events within an interval corresponding to the fixed duration of the time windows (234). The method further comprises obtaining one or more qualified groups of life-style events within the distribution of life-style events, and thereby obtaining a set of qualified groups of life-style events 240, wherein each qualified group of life-style events comprises a group-time indicator. The method further comprises, for each respective qualified group of life-style events within the set of qualified groups of life-style events: (i) determining, on a temporal basis, a subset of grouped life-style data records corresponding to the respective qualified group of life-style events, using the group-time indicator and the relative time of each of the life-style data records in each selected subset of life-style data records of the set of selected subsets of life-style data records, and thereby obtaining a subset of grouped life-style data records, (ii) processing the subset of grouped life-style data records of the respective qualified group of life-style events to obtain display data 249 further configured to represent a measure of central tendency and a measure of variability of life-style events within the respective qualified group of life-style events, wherein the measure of central tendency and the measure of variability is related to the relative time and/or the measure of intensity. The method further comprises communicating the display data 249 to (i) the subject, (ii) to a health care provider, or (iii) to the user of the device 250, and thereby communicating the central tendency and the variability of the injection events.

### List of Embodiments

1. A device (250) for communicating a dose history configured for representing a central tendency and a variability of a distribution of injections with a blood glucose regulating medicament applied by a subject with a treatment regimen (206);
   the device comprises one or more processors (274) and a memory (192/290), the memory storing instructions that, when executed by the one or more processors, perform a method of:
   obtaining a first data set (220) from one or more injection devices used by the subject to apply the treatment regimen (206), the first data set comprising a plurality of medicament records taken over a time course, each respective medicament record (222) in the plurality of medicament records comprising:
      (i) a respective medicament injection event (224) including an amount of medicament (226) injected into the subject using a respective injection device (104) in the one or more injection devices,
      (ii) a corresponding electronic injection event timestamp (229) within the time course that is automatically generated by the respective injection device upon occurrence of the respective medicament injection event (224);
   creating a plurality of consecutive time windows (233) within the time course, wherein each time window (234) is of the same fixed duration,
      for each respective time window (234), obtaining a subset of medicament records (235), and thereby obtaining a plurality of subsets of medicament records, wherein each respective subset of medicament records (235) comprises a number of medicament records from the first data set (220), and wherein each respective medicament record (222) within the respective subset of medicament records (235) have a timestamp (229) in the respective time window (234);
   for each respective medicament record (222), within each subset of medicament records (235) of the plurality of subsets of medicament records, assigning a corresponding relative time (237) being the relative time within the time window (234);
   selecting a set of subsets of medicament records (235) from the plurality of subsets of medicament records and thereby obtaining a set of selected subsets of medicament records (230) comprising a number of selected subsets of medicament records (231) representing a distribution of injection events within an interval corresponding to the fixed duration of the time windows (234);
   obtaining one or more qualified groups of injection events within the distribution of injection events, and thereby obtaining a set of qualified groups of injection events (240), wherein each qualified group of injection events comprises a group-time indicator (243);
   for each respective qualified group of injection events (241) within the set of qualified groups of injection events (240):
      (iii) determining, on a temporal basis, a subset of grouped medicament records (242) corresponding to the respective qualified group of injection events (241), using the group-time indicator (243) and the relative time of each of the medicament records (222) in each selected subset of medicament records (231) of the set of selected subsets of medicament records (230), and thereby obtaining a subset of grouped medicament records,
      (iv) processing the subset of grouped medicament records (242) of the respective qualified group of injection events to obtain display data (249) configured to represent a measure of central tendency and a measure of variability of injection events within the respective qualified group of injection events (241), wherein the measure of central tendency (244, 246) and the measure of variability (245, 247) is related to the relative time (237); and
   communicating the display data (249) to (i) the subject, (ii) to a health care provider, or (iii) to the user of the device (250), and thereby communicating the central tendency and the variability of the injection events.

### Central Tendency and Variability Relating to Amount of Injected Medicament

2. The device according to embodiment 1, wherein the step (ii) of processing the subset of grouped medicament records (242) of the respective qualified group of injection events to obtain display data (249) further comprises:
   processing the subset of grouped medicament records (242) of the respective qualified group of injection events to obtain display data (249) configured to represent a measure of central tendency and a measure of variability of injection events within the respective qualified group of injection events (241), wherein the measure of central tendency (244, 246) and the measure of variability (245, 247) is related to the amount of medicament (226) injected into the body.

### Peak-Time as Group-Time Indicator

3. The device according to any of embodiments 1 or 2, wherein the step of obtaining one or more qualified groups of injection events within the distribution of injection events, and thereby obtaining a set of qualified groups of injection (240) events further comprises:
   estimating a probability density function (310) of the distribution of injections events, by using the set of selected subsets of medicament records (230);
   identifying one or more groups of injection events within the distribution of injection events, by using the probability density function (310), wherein each of the identified groups of injection events (321) are identified by a peak (322) indicating a local maximum of the probability density function (310), wherein the identified peak comprises a peak value (323) and a corresponding peak time (324), and thereby obtaining a set of identified groups of injection events (320);
   responsive to identifying one or more groups of injection events, for each respective identified group of injection events (321) within the set of identified groups of injection events (320), evaluating whether the respective identified group of injection events (321) is qualified to be communicated, and thereby obtaining a set of qualified groups of injection events (240); and
   wherein the step (i) of determining the subset of grouped medicament records (242), for each respective qualified group of injection events (241) further comprises using the peak-time (324) as the group-time indicator (243).
4. The device according to embodiment 3, wherein the step of evaluating whether the respective identified group of injection events is qualified to be communicated comprises evaluating whether a function of the peak value (323) is satisfies a pre-defined threshold for qualification (329).

### Using the Pre-Defined Time Range as Group Indicator

5. The device according to any of embodiments 1 or 2, wherein the step of obtaining one or more qualified groups of injection events within the distribution of injection events, and thereby obtaining a set of qualified groups of injection events (240), further comprises:
   obtaining a set of pre-defined time ranges (330), wherein each of the pre-defined time ranges (331) within the set of pre-defined time ranges (330) are defined within the fixed duration of the time windows, and wherein none of the pre-defined time ranges (331) are overlapping another pre-defined time range (331) within the set of pre-defined time ranges (330);
   for each pre-defined time range (331):
      obtaining an identified group of injection events (321) and thereby obtaining a set of identified groups of injection events (320),
      evaluating whether the identified group of injection events (321) is a qualified group of injection events (241), and thereby obtaining a set of qualified groups of injection events (240); and
   wherein the step (i) of determining the subset of grouped medicament records (242), for each respective qualified group of injections events (241), further comprises using the pre-defined time range (331) as the group-time indicator (243).

### Further Aspects of Qualifying an Identified Group

6. The device according to embodiment 5, wherein, for each pre-defined time range (331), the step of obtaining an identified group of injection events (321) further comprises:
   determining, on a temporal basis, a subset of identified medicament records (332) of the medicament records (222) within the set of selected subsets of medicament records (230), wherein the medicament records (222) within the subset of identified medicament records (332) have a relative time (237) within the respective pre-defined time range (331); and
   wherein the step of evaluating whether the identified group of injection events (321) is a qualified group of injection events (241) further comprises:
   evaluating the number of medicament records within the subset of identified medicament records (333),
   evaluating whether the number of medicament records (333) is above a pre-defined threshold (329), wherein the identified group (221) is qualified, or below a pre-defined threshold (329), wherein the identified group (221) is disqualified;
   responsive to the identified group (221) is qualified characterizing the identified group of injection events as a qualified group of injection events (241).

### Further Aspect of Using User Input for Qualifying

7. The device according to embodiment 5, wherein, for each pre-defined time range (331), the step of evaluating whether the identified group of injection events (321) is a qualified group of injection events (241) further comprises:
   obtaining a user input (322) indicating whether the pre-defined time range (331) is qualified or disqualified; and
   responsive to the obtained user input indicates that the pre-defined time range (331) is qualified, characterizing the identified group of injection events as a qualified group of injection events.

### Further Aspect of Using Pre-Defined Input for Qualifying

8. The device according to embodiment 5, wherein, for each pre-defined time range (331), the step of evaluating whether the identified group of injection events (321) is a qualified group of injection events (241) further comprises:
   obtaining a pre-defined input indicating whether the pre-defined time range (331) is qualified or disqualified; and
   responsive to the obtained pre-defined input indicates that the pre-defined time range (331) is qualified, characterizing the identified group of injection events as a qualified group of injection events.

### Further Aspects of Central Tendency and Measure of Variability

9. The device according to any of the previous embodiments, wherein the step (ii) of processing the subset of grouped medicament records, for each respective qualified group of injection events, to obtain display data configured to represent a central tendency and a variability of injection events comprises:
   evaluating a measure of central tendency for the relative time (237) and the amount of medicament (226) injected into the body, and
   evaluating a measure of variability for the relative time (237) and the amount of medicament (226) injected into the body.
10. The device according to embodiment 9, wherein the step of evaluating a measure of central tendency comprises evaluating a median, and determining a measure of variability comprises evaluating an upper and a lower percentile.
11. The device according to embodiment 9, wherein the step of evaluating a measure of central tendency comprises evaluating a mean, and determining a measure of variability comprises evaluating a standard deviation.

### Further Aspects of Display Data

12. The device according to any of the previous embodiments, wherein the display data comprises the data configured to represent an average and a variability of injection events within the respective qualified group of injection events.
13. The invention according to any of the previous embodiments wherein the display data comprises the set of qualified groups of injection events.

### Further Aspects of Displaying Average and Variability as a Polygon

14. The device according to any of the previous embodiments, wherein the step (ii) of processing the subset of grouped medicament records (242), for each respective qualified group of injection events (241), to obtain display data (249), further comprises:
   associating with each qualified group of injection events (241):
      a shape data structure (350), configured for representing the central tendency and the variability of the subset of grouped medicament records (242) corresponding to the respective qualified group of injection events (241), wherein the shape data structure (350) comprises:
         a central tendency data-structure (351) comprising a central tendency polygon (352) configured for visualizing a polygon with a two-dimensional shape indicating the measure of central tendency,
         a variability data-structure (353) comprising a variability polygon (354) configured for visualizing a polygon with a two-dimensional shape identifying the measure of variability.

### Further Aspects of Communicating in a Table

15. The device according to embodiment 10, wherein the step (ii) of processing the subset of grouped medicament records (242), for each respective qualified group of injection events (241), to obtain display data (249), further comprises:
   associating with each qualified group of injection events (241) a table data structure (354) comprising:
      a qualified group identification (355),
      a median time (356) and a time variation (357) based on the relative time (237), of the subset of grouped medicament records (242), and
      a median dose (358) and a dose variation (359) based on the amount of injected medicament (226) of the subset of grouped medicament records (242).
         Further aspects of enabling an indication of the frequency of injections in the respective qualified group of injection events
16. The device according to any of the previous embodiments, wherein the step (ii) of processing the subset of grouped medicament records (242), for each respective qualified group of injection events (241), to obtain display data (249), further comprises:
   obtaining display data (249) configured to represent a frequency indicator (334) indicating the frequency of an injection event of the respective qualified group of injection events (241), wherein the frequency indicator (334) is a function of:
      the number of medicament records in the subset of grouped medicament records (242) over the total number of medicament records in all subsets of grouped medicament records in the set of qualified groups of injection events (240), or
      the number of medicament records in the subset of grouped medicament records (242) over the total number of medicament records of all selected subsets medicament records (231) in the set of selected subsets of medicament records (230);
   thereby enabling an indication on the frequency of injections in the respective qualified group of injection events (242), and wherein the display data (249) further comprises the frequency indicator (334).

### Further Aspects of Enabling an Indication on the Frequency of Injections in the Respective Qualified Group of Injection Events Using the Normalized Probability Distribution

17. The device according to any of embodiments 3 or 4, wherein the step (ii) of processing the subset of grouped medicament records (242), for each respective qualified group of injection events (241), to obtain display data (249), further comprises:
   obtaining display data (249) configured to indicate a frequency of an injection event of the respective qualified group of injection events (241);
   normalizing the probability density function (310) and the peak value to obtain a normalized peak value (325), whereby the normalized peak value (325) indicates the frequency of an injection event of the respective qualified group of injection events (241), and wherein the display data (249) comprises the normalized peak value (325).

### GLP-1

18. The device according to any of the previous embodiments, wherein the treatment regimen (206) comprises a GLP-1 receptor agonist dosage regimen (216), with a medicament comprising a GLP-1 receptor agonist.

### Bolus Insulin

19. The device according to any of the previous embodiments, wherein the treatment regimen comprises a bolus insulin medicament dosage regimen (208) with a short acting insulin medicament (210).

### Medicament Type

20. The device according to any of the previous embodiments, wherein each respective medicament record (222) in the plurality of medicament records further comprises:
   (iii) a corresponding type of medicament (228) injected into the subject; and
   wherein the set of selected subsets of medicament records comprises the same type of medicament (228) and thereby represents a distribution of injection events corresponding to the respective type of medicament; and
   wherein the step (ii) of processing the subset of grouped medicament records (242), for each respective qualified group of injection events (241), to obtain display data (249), further comprises obtaining display data (249) configured to represent the respective type of medicament (228).

### Further Aspects of Medicament Type: Bolus and Basal

21. The device according to embodiment 20, wherein the treatment regimen comprises a bolus insulin medicament dosage regimen with a short acting insulin medicament and a basal insulin medicament dosage regimen with a long acting insulin medicament.

### Further Aspects of Display for Injection Data—First Coordinate System

22. The device according to any of the previous embodiments, wherein the device further comprises a display (282) configured for representing a first coordinate system (710), and wherein the step of communicating display data further comprises:
   displaying the obtained display data (249), configured to represent a central tendency and a variability of injection events within the respective qualified group of injection events, in the first coordinate system (710) on the display (282), the first coordinate system comprises a first axis (711) and a second axis (712):
   wherein the first coordinate system is adapted to represent the central tendency and the variability of the injection events; and
   wherein the first axis represent the relative time (237) and are defined within the interval defined by the time window (234), and wherein the second axis (712) represents the amount of injected medicament (226).

### Further Aspects of Injection Data and Blood Glucose Data

23. The device according to any of the embodiments 1-21, wherein the method further comprises:
   obtaining a second data set (340), wherein the second data set (340) comprises a plurality of autonomous glucose measurements of the subject within the time course and, for each respective autonomous glucose measurement (341) in the plurality of autonomous glucose measurements, a glucose measurement timestamp (342) representing when the respective measurement was made; and
   for each respective time window (234), creating a set of glucose measurements (345), and thereby creating a plurality of sets of glucose measurements, and wherein each glucose measurement (341) within the respective set of glucose measurements (345) have a timestamp (342) in the respective time window (234);
   for each respective glucose measurement (311), associating a corresponding relative time (343) being the relative time within the time window, whereby the plurality of sets of glucose measurements are representing a distribution of glucose measurements within the time window;
   calculating, for the plurality of sets of glucose measurements, the central tendency and the variability as a function of the relative time,
   wherein the display data further comprises the plurality of sets of glucose measurements, the corresponding relative time, and the calculated central tendency and the variability as a function of the relative time.

### Further Aspects of Display for Injection Data and Blood Glucose Data—First and Second Coordinate System

24. The device according to embodiment 23, wherein the display (282) is adapted to represent a first (710) and a second coordinate system (720) each comprising a first axis (711, 721) and a second axis (712, 722), and
   wherein the first coordinate system (710) is adapted to represent the central tendency and the variability of the injection events; and
   wherein the second coordinate system is adapted to represent the central tendency and the variability of the glucose data; and
   wherein the step of communicating display data further comprises:
   displaying the obtained display data (249), configured to represent an central tendency and a variability of injection events within a respective qualified group of injection events, in the first coordinate system (710) on the display (282), and
   displaying the obtained display data (249), comprising an central tendency and a variability of the plurality of sets of glucose measurements as a function of time, in the second coordinate system (720) on the display (282), and
   wherein, for the first coordinate system (710), the second axis (712) represents the amount of injected medicament (226), and wherein, for the second coordinate system (720), the second axis (722) represents a blood glucose concentration, and wherein the first axis (711, 721) of each coordinate systems (710, 720) represent the relative time (237, 343) and are defined within the interval defined by the time window (234).

### Further Aspects of Life-Style Event

25. The device according to any of the previous embodiments, further adapted for communicating a life-style event history representing an central tendency and a variability of a distribution of life-style related events within the time course, which the subject has engaged in, wherein the method further comprises:
   obtaining a third data set from one or more wearable life-style measurement devices (103) used by the subject to acquire life-style data, the third data set comprises a plurality of life-style data records over the time course, each respective life-style data record in the plurality of life-style data records comprises:
      (i) a respective life-style event including a measure of intensity indicating the effect on the subject using the respective measurement device (103)
      (ii) a corresponding electronic life-style event timestamp within the time course that is automatically generated by the respective life-style measurement device (103) upon occurrence of the respective life-style related event, or by user actuation of the respective life-style measurement device, or a begin timestamp and an end timestamp indicating the beginning and the ending time of the life-style event engaged in by the subject;
      for each respective time window (234), obtaining a subset of life-style data records, and thereby obtaining a plurality of subsets of life-style data records, wherein each respective subset of life-style data records comprises a number of life-style data records from the third data set, and wherein each respective life-style data record within the respective subset of life-style data records have a timestamp in the respective time window (234);
   for each respective life-style data record, within each subset of life-style data records of the plurality of subsets of life-style data records, assigning a corresponding relative time (237) being the relative time within the time window (234);
   selecting a set of subsets of life-style data records from the plurality of subsets of life-style data records and thereby obtaining a set of selected subsets of life-style data records comprising a number of selected subsets of life-style data records representing a distribution of life-style events within an interval corresponding to the fixed duration of the time windows (234);
   obtaining one or more qualified groups of life-style events within the distribution of life-style events, and thereby obtaining a set of qualified groups of life-style events (240), wherein each qualified group of life-style events comprises a group-time indicator;
   for each respective qualified group of life-style events within the set of qualified groups of life-style events:
      (i) determining, on a temporal basis, a subset of grouped life-style data records corresponding to the respective qualified group of life-style events, using the group-time indicator and the relative time of each of the life-style data records in each selected subset of life-style data records of the set of selected subsets of life-style data records, and thereby obtaining a subset of grouped life-style data records,
      (ii) processing the subset of grouped life-style data records of the respective qualified group of life-style events to obtain display data (249) further configured to represent a measure of central tendency and a measure of variability of life-style events within the respective qualified group of life-style events, wherein the measure of central tendency and the measure of variability is related to the relative time and/or the measure of intensity; and
communicating the display data (249) to (i) the subject, (ii) to a health care provider, or (iii) to the user of the device (250), and thereby communicating the central tendency and the variability of the injection events.
26. A method for communicating a dose event history representing a central tendency and a variability of a distribution of a distribution injections with a blood glucose regulating medicament applied by a subject with a treatment regimen (206);
   using a device (250) comprising one or more processors (274) and a memory (192/290), the memory storing instructions that, when executed by the one or more processors, perform a method of:
   obtaining a first data set (220) from one or more injection devices used by the subject to apply the treatment regimen (206), the first data set comprising a plurality of medicament records taken over a time course, each respective medicament record (222) in the plurality of medicament records comprising:
      (i) a respective medicament injection event (224) including an amount of medicament (226) injected into the subject using a respective injection device (104) in the one or more injection devices,
      (ii) a corresponding electronic injection event timestamp (229) within the time course that is automatically generated by the respective injection device upon occurrence of the respective medicament injection event (224);
   creating a plurality of consecutive time windows (233) within the time course, wherein each time window (234) is of the same fixed duration,
      for each respective time window (234), obtaining a subset of medicament records (235), and thereby obtaining a plurality of subsets of medicament records, wherein each respective subset of medicament records (235) comprises a number of medicament records from the first data set (220), and wherein each respective medicament record (222) within the respective subset of medicament records (235) have a timestamp (229) in the respective time window (234);
   for each respective medicament record (222), within each subset of medicament records (235) of the plurality of subsets of medicament records, assigning a corresponding relative time (237) being the relative time within the time window (234);
   selecting a set of subsets of medicament records (235) from the plurality of subsets of medicament records and thereby obtaining a set of selected subsets of medicament records (230) comprising a number of selected subsets of medicament records (231) representing a distribution of injection events within an interval corresponding to the fixed duration of the time windows (234);
   obtaining one or more qualified groups of injection events within the distribution of injection events, and thereby obtaining a set of qualified groups of injection events (240), wherein each qualified group of injection events comprises a group-time indicator (243);
   for each respective qualified group of injection events (241) within the set of qualified groups of injection events (240):
      (i) determining, on a temporal basis, a subset of grouped medicament records (242) corresponding to the respective qualified group of injection events (241), using the group-time indicator (243) and the relative time of each of the medicament records (222) in each selected subset of medicament records (231) of the set of selected subsets of medicament records (230), and thereby obtaining a subset of grouped medicament records,
      (ii) processing the subset of grouped medicament records (242) of the respective qualified group of injection events to obtain display data (249) configured to represent a measure of central tendency and a measure of variability of injection events within the respective qualified group of injection events (241), wherein the measure of central tendency (244, 246) and the measure of variability (245, 247) is related to the relative time (237); and
   communicating the display data (249) to (i) the subject, (ii) to a health care provider, or (iii) to the user of the device (250), and thereby communicating the central tendency and the variability of the injection events.
27. A computer program comprising instructions that, when executed by a computer having one or more processors and a memory, perform the method of embodiment 26.
28. A computer-readable data carrier having stored thereon the computer program according to embodiment 14.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a nontransitory computer readable storage medium. For instance, the computer program product could contain the program modules shown in any combination of FIGS. 1, 2, 3 and/or described in FIG. 4. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A device for communicating a dose history configured for representing a central tendency and a variability of a distribution of injections with a blood glucose regulating medicament applied by a subject with a treatment regimen;
the device comprises one or more processors and a memory, the memory storing instructions that, when executed by the one or more processors, perform a method of:
obtaining a first data set from one or more injection devices used by the subject to apply the treatment regimen, the first data set comprising a plurality of medicament records taken over a time course, each respective medicament record in the plurality of medicament records comprising:
(i) a respective medicament injection event including an amount of medicament injected into the subject using a respective injection device in the one or more injection devices,
(ii) a corresponding electronic injection event timestamp within the time course that is automatically generated by the respective injection device upon occurrence of the respective medicament injection event;
creating a plurality of consecutive time windows within the time course, wherein each time window is of the same fixed duration,
for each respective time window, obtaining a subset of medicament records, and thereby obtaining a plurality of subsets of medicament records, wherein each respective subset of medicament records comprises a number of medicament records from the first data set, and wherein each respective medicament record within the respective subset of medicament records have a timestamp in the respective time window for each respective medicament record, within each subset of medicament records of the plurality of subsets of medicament records, assigning a corresponding relative time being the relative time within the time window;
selecting a set of subsets of medicament records from the plurality of subsets of medicament records and thereby obtaining a set of selected subsets of medicament records comprising a number of selected subsets of medicament records representing a distribution of injection events within an interval corresponding to the fixed duration of the time windows;
obtaining one or more qualified groups of injection events within the distribution of injection events, and thereby obtaining a set of qualified groups of injection events, wherein each qualified group of injection events comprises a group-time indicator;
for each respective qualified group of injection events within the set of qualified groups of injection events:
(i) determining, on a temporal basis, a subset of grouped medicament records corresponding to the respective qualified group of injection events using the group-time indicator and the relative time of each of the medicament records in each selected subset of medicament records of the set of selected subsets of medicament records, and thereby obtaining a subset of grouped medicament records,
(ii) processing the subset of grouped medicament records of the respective qualified group of injection events and evaluating a measure of central tendency and a measure of variability of the subset of grouped medicament records to obtain display data configured to represent a measure of central tendency and a measure of variability of injection events within the respective qualified group of injection events, wherein the measure of central tendency and the measure of variability is related to the relative time; and
communicating the display data comprising the evaluated measure of central tendency and the evaluated measure of variability of the subset of grouped medicament records to (i) the subject, (ii) to a health care provider, or (iii) to the user of the device, and thereby communicating the central tendency and the variability of the distribution of the injection events representing the set of selected subsets of medicament records.

2. The device according to claim 1, wherein the step (ii) of processing the subset of grouped medicament records of the respective qualified group of injection events to obtain display data further comprises:
processing the subset of grouped medicament records of the respective qualified group of injection events to obtain display data configured to represent a measure of central tendency and a measure of variability of injection events within the respective qualified group of injection events, wherein the measure of central tendency and the measure of variability is related to the amount of medicament injected into the body.

3. The device according to claim 1, wherein the step of obtaining one or more qualified groups of injection events within the distribution of injection events, and thereby obtaining a set of qualified groups of injection events further comprises:
estimating a probability density function of the distribution of injections events, by using the set of selected subsets of medicament records;
identifying one or more groups of injection events within the distribution of injection events, by using the probability density function, wherein each of the identified groups of injection events are identified by a peak indicating a local maximum of the probability density function, wherein the identified peak comprises a peak value and a corresponding peak time, and thereby obtaining a set of identified groups of injection events;
responsive to identifying one or more groups of injection events, for each respective identified group of injection events within the set of identified groups of injection events, evaluating whether the respective identified group of injection events is qualified to be communicated, comprising evaluating a function of the peak value, and deeming the respective identified group qualified to be communicated, when the evaluated function of the peak value is above a pre-defined threshold for qualification, and thereby obtaining a set of qualified groups of injection events; and
wherein the step (i) of determining the subset of grouped medicament records for each respective qualified group of injection events further comprises using the peak-time as the group-time indicator.

4. The device according to claim 3, further comprising:
for each respective qualified group of injection events, obtaining display data further configured to indicate a frequency of an injection event of the respective qualified group of injection events;
normalizing the probability density function and the peak value to obtain a normalized peak value, whereby the normalized peak value indicates the frequency of an injection event of the respective qualified group of injection events, and wherein the display data further comprises the normalized peak value.

5. The device according to claim 1, wherein the step of obtaining one or more qualified groups of injection events within the distribution of injection events, and thereby obtaining a set of qualified groups of injection events, further comprises:
obtaining a set of pre-defined time ranges, wherein each of the pre-defined time ranges within the set of pre-defined time ranges are defined within the fixed duration of the time windows, and wherein none of the pre-defined time ranges are overlapping another pre-defined time range within the set of pre-defined time ranges;
for each pre-defined time range:
obtaining an identified group of injection events and thereby obtaining a set of identified groups of injection events,
evaluating whether the identified group of injection events is a qualified group of injection events, and thereby obtaining a set of qualified groups of injection events; and
wherein the step (i) of determining the subset of grouped medicament records for each respective qualified group of injections events, further comprises using the pre-defined time range as the group-time indicator.

6. The device according to claim 5, wherein, for each pre-defined time range, the step of obtaining an identified group of injection events further comprises:
determining, on a temporal basis, a subset of identified medicament records of the medicament records within the set of selected subsets of medicament records, wherein the medicament records within the subset of identified medicament records have a relative time within the respective pre-defined time range; and
wherein the step of evaluating whether the identified group of injection events is a qualified group of injection events further comprises:
evaluating the number of medicament records within the subset of identified medicament records,
evaluating whether the number of medicament records is above a pre-defined threshold wherein the identified group is deemed qualified, or below a pre-defined threshold, wherein the identified group is deemed disqualified;
responsive to the identified group is deemed qualified characterizing the identified group of injection events as a qualified group of injection events.

7. The device according to claim 1, wherein the step (ii) of processing the subset of grouped medicament records, for each respective qualified group of injection events, to obtain display data, further comprises:
associating with each qualified group of injection events:
a shape data structure, configured for representing the central tendency and the variability of the subset of grouped medicament records corresponding to the respective qualified group of injection events, wherein the shape data structure comprises:
a central tendency data-structure comprising a central tendency polygon configured for visualizing a polygon with a two-dimensional shape indicating the measure of central tendency,
a variability data-structure comprising a variability polygon configured for visualizing a polygon with a two-dimensional shape identifying the measure of variability.

8. The device according to claim 1, wherein the step (ii) of processing the subset of grouped medicament records, for each respective qualified group of injection events, to obtain display data, further comprises:
obtaining display data configured to represent a frequency indicator indicating the frequency of an injection event of the respective qualified group of injection events, wherein the frequency indicator is a function of:
the number of medicament records in the subset of grouped medicament records over the total number of medicament records in all subsets of grouped medicament records in the set of qualified groups of injection events, or
the number of medicament records in the subset of grouped medicament records over the total number of medicament records of all selected subsets medicament records in the set of selected subsets of medicament records;
thereby enabling an indication on the frequency of injections in the respective qualified group of injection events, and wherein the display data further comprises the frequency indicator.

9. The device according to claim 1, wherein each respective medicament record in the plurality of medicament records further comprises:
(iii) a corresponding type of medicament injected into the subject; and
wherein the set of selected subsets of medicament records comprises the same type of medicament and thereby represents a distribution of injection events corresponding to the respective type of medicament; and
wherein the step (ii) of processing the subset of grouped medicament records for each respective qualified group of injection events, to obtain display data, further comprises obtaining display data configured to represent the respective type of medicament.

10. The device according to claim 9, wherein the treatment regimen comprises a bolus insulin medicament dosage regimen with a short acting insulin medicament and a basal insulin medicament dosage regimen with a long acting insulin medicament.

11. The device according to claim 1, wherein the method further comprises:
obtaining a second data set wherein the second data set comprises a plurality of autonomous glucose measurements of the subject within the time course and, for each respective autonomous glucose measurement in the plurality of autonomous glucose measurements, a glucose measurement timestamp representing when the respective measurement was made; and
for each respective time window creating a set of glucose measurements, and thereby creating a plurality of sets of glucose measurements, and wherein each glucose measurement within the respective set of glucose measurements have a timestamp in the respective time window;
for each respective glucose measurement, associating a corresponding relative time being the relative time within the time window, whereby the plurality of sets of glucose measurements are representing a distribution of glucose measurements within the time window;
calculating, for the plurality of sets of glucose measurements, the central tendency and the variability as a function of the relative time,
wherein the display data further comprises the plurality of sets of glucose measurements, the corresponding relative time, and the calculated central tendency and the variability as a function of the relative time.

12. The device according to claim 11, wherein the display is adapted to represent a first and a second coordinate system each comprising a first axis and a second axis, and
wherein the first coordinate system is adapted to represent the central tendency and the variability of the injection events; and
wherein the second coordinate system is adapted to represent the central tendency and the variability of the glucose data; and
wherein the step of communicating display data further comprises:
displaying the obtained display data, configured to represent an central tendency and a variability of injection events within a respective qualified group of injection events, in the first coordinate system on the display, and
displaying the obtained display data, comprising an central tendency and a variability of the plurality of sets of glucose measurements as a function of time, in the second coordinate system on the display, and
wherein, for the first coordinate system, the second axis represents the amount of injected medicament, and wherein, for the second coordinate system the second axis represents a blood glucose concentration, and wherein the first axis of each coordinate systems represent the relative time and are defined within the interval defined by the time window.

13. The device according to claim 1, further adapted for communicating a life-style event history representing an central tendency and a variability of a distribution of life-style related events within the time course, which the subject has engaged in, wherein the method further comprises:
obtaining a third data set from one or more wearable life-style measurement devices used by the subject to acquire life-style data, the third data set comprises a plurality of life-style data records over the time course, each respective life-style data record in the plurality of life-style data records comprises:
(i) a respective life-style event including a measure of intensity indicating the effect on the subject using the respective measurement device
(ii) a corresponding electronic life-style event timestamp within the time course that is automatically generated by the respective life-style measurement device upon occurrence of the respective life-style related event, or by user actuation of the respective life-style measurement device, or a begin timestamp and an end timestamp indicating the beginning and the ending time of the life-style event engaged in by the subject;
for each respective time window, obtaining a subset of life-style data records, and thereby obtaining a plurality of subsets of life-style data records, wherein each respective subset of life-style data records comprises a number of life-style data records from the third data set, and wherein each respective life-style data record within the respective subset of life-style data records have a timestamp in the respective time window;
for each respective life-style data record, within each subset of life-style data records of the plurality of subsets of life-style data records, assigning a corresponding relative time being the relative time within the time window;
selecting a set of subsets of life-style data records from the plurality of subsets of life-style data records and thereby obtaining a set of selected subsets of life-style data records comprising a number of selected subsets of life-style data records representing a distribution of life-style events within an interval corresponding to the fixed duration of the time windows;
obtaining one or more qualified groups of life-style events within the distribution of life-style events, and thereby obtaining a set of qualified groups of life-style events, wherein each qualified group of life-style events comprises a group-time indicator;
for each respective qualified group of life-style events within the set of qualified groups of life-style events:
(i) determining, on a temporal basis, a subset of grouped life-style data records corresponding to the respective qualified group of life-style events, using the group-time indicator and the relative time of each of the life-style data records in each selected subset of life-style data records of the set of selected subsets of life-style data records, and thereby obtaining a subset of grouped life-style data records,
(ii) processing the subset of grouped life-style data records of the respective qualified group of life-style events to obtain display data further configured to represent a measure of central tendency and a measure of variability of life-style events within the respective qualified group of life-style events, wherein the measure of central tendency and the measure of variability is related to the relative time and/or the measure of intensity; and
communicating the display data to (i) the subject, (ii) to a health care provider, or (iii) to the user of the device, and thereby communicating the central tendency and the variability of the injection events.

14. A method for communicating a dose event history representing a central tendency and a variability of a distribution of a distribution injections with a blood glucose regulating medicament applied by a subject with a treatment regimen;
using a device comprising one or more processors and a memory, the memory storing instructions that, when executed by the one or more processors, perform a method of:
obtaining a first data set from one or more injection devices used by the subject to apply the treatment regimen, the first data set comprising a plurality of medicament records taken over a time course, each respective medicament record in the plurality of medicament records comprising:
(i) a respective medicament injection event including an amount of medicament injected into the subject using a respective injection device in the one or more injection devices,
(ii) a corresponding electronic injection event timestamp within the time course that is automatically generated by the respective injection device upon occurrence of the respective medicament injection event;
creating a plurality of consecutive time windows within the time course, wherein each time window is of the same fixed duration,
for each respective time window, obtaining a subset of medicament records, and thereby obtaining a plurality of subsets of medicament records, wherein each respective subset of medicament records comprises a number of medicament records from the first data set, and wherein each respective medicament record within the respective subset of medicament records have a timestamp in the respective time window;
for each respective medicament record, within each subset of medicament records of the plurality of subsets of medicament records, assigning a corresponding relative time being the relative time within the time window;
selecting a set of subsets of medicament records from the plurality of subsets of medicament records and thereby obtaining a set of selected subsets of medicament records comprising a number of selected subsets of medicament records representing a distribution of injection events within an interval corresponding to the fixed duration of the time windows;
obtaining one or more qualified groups of injection events within the distribution of injection events, and thereby obtaining a set of qualified groups of injection events, wherein each qualified group of injection events comprises a group-time indicator;
for each respective qualified group of injection events within the set of qualified groups of injection events:
(iii) determining, on a temporal basis, a subset of grouped medicament records corresponding to the respective qualified group of injection events, using the group-time indicator and the relative time of each of the medicament records in each selected subset of medicament records of the set of selected subsets of medicament records, and thereby obtaining a subset of grouped medicament records,
(iv) processing the subset of grouped medicament records of the respective qualified group of injection events and evaluating a measure of central tendency and a measure of variability of the subset of grouped medicament records to obtain display data configured to represent a measure of central tendency and a measure of variability of injection events within the respective qualified group of injection events, wherein the measure of central tendency and the measure of variability is related to the relative time; and
communicating the display data comprising the evaluated measure of central tendency and the evaluated measure of variability of the subset of grouped medicament records to (i) the subject, (ii) to a health care provider, or (iii) to the user of the device, and thereby communicating the central tendency and the variability of the distribution of the injection events representing the set of selected subsets of medicament records.

15. A computer program comprising instructions that, when executed by a computer having one or more processors and a memory, perform the method of claim 14.
